(19) United States
(12) Patent Application Publication
(10) Pub. No.: US 2025/0024752 A1
(43) Pub. Date: Jan. 16, 2025

(54) PHOTOELECTRIC CONVERSION ELEMENT, IMAGING ELEMENT, OPTICAL SENSOR, AND COMPOUND

(71) Applicant: FUJIFILM Corporaton, Tokyo (JP)

(72) Inventors: Masaki MORITA, Kanagawa (JP); Saika IZUMI, Kanagawa (JP); Yasunori YONEKUTA, Kanagawa (JP)

(73) Assignee: FUJIFILM Corporaton, Tokyo (JP)

(21) Appl. No.: 18/827,468

(22) Filed: Sep. 6, 2024

Related U.S. Application Data
(63) Continuation of application No. PCT/JP2023/010110, filed on Mar. 15, 2023.

(30) Foreign Application Priority Data
Mar. 31, 2022 (JP) 2022-058218

(51) Int. Cl. H10K 85/60; C07D 237/26; C07D 401/06; C07D 403/06; C07D 413/06; C07D 487/04; C07D 491/048; C07D 495/04; C07D 495/14; C07D 513/04; C07D 519/00; H10K 30/30

(52) U.S. Cl. CPC H10K 85/6572; C07D 237/26; C07D 401/06; C07D 403/06; C07D 413/06; C07D 487/04; C07D 491/048; C07D 495/04; C07D 495/14; C07D 513/04; C07D 519/00; H10K 85/654; H10K 85/657; H10K 85/6576; H10K 30/30

(57) ABSTRACT

An object of the present invention is to provide a photoelectric conversion element which has excellent manufacturing suitability, an imaging element, an optical sensor, and a compound. A photoelectric conversion element according to the present invention including in the following order, a conductive film, a photoelectric conversion film, and a transparent conductive film, in which the photoelectric conversion film contains a compound represented by Formula (1).

### CROSS-REFERENCE TO RELATED APPLICATIONS

This application is a Continuation of PCT International Application No. PCT/JP2023/010110 filed on Mar. 15, 2023, which claims priority under 35 U.S.C. § 119 (a) to Japanese Patent Application No. 2022-058218 filed on Mar. 31, 2022. The above applications are hereby expressly incorporated by reference, in their entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, development of an element having a photoelectric conversion film has progressed. For example, WO2020/013246A discloses a compound applied to a photoelectric conversion element.

### SUMMARY OF THE INVENTION

In recent years, along with the demand for improving the performance of imaging elements, optical sensors, and the like, further improvements are required for various characteristics required for photoelectric conversion elements used therein.

For example, the photoelectric conversion element is required to have excellent manufacturing suitability such that the photoelectric conversion efficiency does not deteriorate even in a case where the vapor deposition rate during the formation of the photoelectric conversion film is increased, in order to meet the requirements for manufacturing a product.

As a result of studying the photoelectric conversion element using the compound disclosed in WO2020/013246A and the like, the present inventor has found that the photoelectric conversion element cannot satisfy the recent demand for manufacturing suitability and further improvement is required.

Therefore, an object of the present invention is to provide a photoelectric conversion element which has excellent manufacturing suitability. In addition, another object of the present invention is to provide an imaging element, an optical sensor, and a compound.

The inventors of the present invention have conducted extensive studies on the above-described problems. As a result, the inventors have found that it is possible to solve the above-described problems by applying the compound having a predetermined structure to the photoelectric conversion film, and have completed the present invention.

[1]

A photoelectric conversion element comprising in the following order, a conductive film, a photoelectric conversion film, and a transparent conductive film, in which the photoelectric conversion film contains a compound represented by Formula (1).

[2]

The photoelectric conversion element according to [1], in which D¹ is a group represented by Formula (D-1).

[3]

The photoelectric conversion element according to [2], in which Ar^{d11} is a group represented by any of Formulae (Ar-1) to (Ar-9).

[4]

The photoelectric conversion element according to [2] or [3], in which Ar^{d11} is a group represented by Formula (Ar-10).

[5]

The photoelectric conversion element according to any one of [1] to [4], in which Z¹¹, Z¹², Z²¹, Z²², and Z³¹ are each independently an oxygen atom or a sulfur atom.

[6]

A photoelectric conversion element comprising in the following order, a conductive film, a photoelectric conversion film, and a transparent conductive film, in which the photoelectric conversion film contains a compound represented by Formula (2).

[7]

The photoelectric conversion element according to [6], in which Ar^{d21} is a group represented by Formula (Ar-10).

[8]

The photoelectric conversion element according to [6] or [7], in which Z¹¹, Z¹², Z²¹, Z²², Z³¹, Z⁴¹, and Z⁴² are each independently an oxygen atom or a sulfur atom.

[9]

The photoelectric conversion element according to any one of [1] to [8], further comprising one or more interlayers between the conductive film and the transparent conductive film, in addition to the photoelectric conversion film.

[10]

An imaging element comprising the photoelectric conversion element according to any one of [1] to [9].

[11]

An optical sensor comprising the photoelectric conversion element according to any one of [1] to [9].

[12]

A compound represented by Formula (1).

[13]

The compound according to [12], in which D¹ is a group represented by Formula (D-1).

[14]

The compound according to [13], in which Ar^{d11} is a group represented by any of Formulae (Ar-1) to (Ar-9).

[15]

The compound according to or [14], in which Ar^{d11} is a group represented by Formula (Ar-10).

[16]

The compound according to any one of to [15], in which Z¹¹, Z¹², Z²¹, Z²², and Z³¹ each independently represent an oxygen atom or a sulfur atom.

[17]

A compound represented by Formula (2).

[18]

The compound according to [17], in which Ar^{d21} is a group represented by Formula (Ar-10).

[19]

The compound according to or [18], in which Z¹¹, Z¹², Z²¹, Z²², Z³¹, Z⁴¹, and Z⁴² are each independently an oxygen atom or a sulfur atom.

According to the present invention, it is possible to provide a photoelectric conversion element having excellent manufacturing suitability. In addition, according to the present invention, it is possible to provide an imaging element, an optical sensor, and a compound.

### BRIEF DESCRIPTION OF THE DRAWINGS

FIG. 1 is a schematic cross-sectional view illustrating a configuration example of a photoelectric conversion element.

FIG. 2 is a schematic cross-sectional view illustrating a configuration example of the photoelectric conversion element.

### DESCRIPTION OF THE PREFERRED EMBODIMENTS

Hereinafter, embodiments of the photoelectric conversion element according to the present invention will be described in detail.

In the present specification, the numerical range represented by “to” means a range including numerical values denoted before and after “to” as a lower limit value and an upper limit value.

In the present specification, a hydrogen atom may be a light hydrogen atom (an ordinary hydrogen atom) or a deuterium atom (for example, a double hydrogen atom and the like).

In the present specification, in a case where there are plural substituents, linking groups, and the like (hereinafter, referred to as “substituents and the like”) represented by specific symbols, or a case where a plurality of substituents and the like are specified all together, each of the substituents and the like may be the same or may be different from each other. This also applies to a case of specifying the number of substituents and the like.

In the present specification, a “substituent” includes a group exemplified by a substituent W described later, unless otherwise specified.

### (Substituent W)

A substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heteroaryl group (a heterocyclic group), a cyano group, a nitro group, an alkoxy group, an aryloxy group, a silyloxy group, a heterocyclic oxy group, an acyloxy group, a carbamoyloxy group, an alkoxycarbonyloxy group, an aryloxycarbonyloxy group, a secondary or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a silyl group, a carboxy group, a phosphoric acid group, a sulfonic acid group, a hydroxy group, a thiol group, an acylamino group, a carbamoyl group, a ureido group, a boronic acid group, and a primary amino group. Each of the above-described groups may further have a substituent (for example, one or more groups of each of the above-described groups, and the like), as possible. For example, an alkyl group which may have a substituent is also included as a form of the substituent W.

In addition, in a case where the substituent W has a carbon atom, the number of carbon atoms of the substituent W is, for example, 1 to 20.

The number of atoms other than a hydrogen atom included in the substituent W is, for example, 1 to 30.

In addition, the specific compound described later preferably does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In addition, examples of the substituent W also include a group including a group represented by A¹, and a 1,3-dicarbonyl ring group. Examples of the 1,3-dicarbonyl ring group include a 1,3-indandione ring group, a 1,3-cyclohexanedione ring group, a 5,5-dimethyl-1,3-cyclohexanedione ring group, and a 1,3-dioxane-4,6-dione ring group.

In the present specification, examples of the halogen atom include a fluorine atom, a chlorine atom, a bromine atom, and an iodine atom.

In the present specification, unless otherwise specified, the number of carbon atoms of the alkyl group is preferably 1 to 20, more preferably 1 to 10, and still more preferably 1 to 6.

The alkyl group may be any of linear, branched, or cyclic.

Examples of the alkyl group include a methyl group, an ethyl group, a n-propyl group, an i-propyl group, a n-butyl group, a t-butyl group, a n-hexyl group, a cyclopentyl group, and the like.

In addition, the alkyl group may be any of a cycloalkyl group, a bicycloalkyl group, or a tricycloalkyl group, and may have a cyclic structure thereof as a partial structure.

In the alkyl group which may have a substituent, examples of a substituent which can be contained in the alkyl group include a group exemplified by the substituent W, and an aryl group (preferably having 6 to 18 carbon atoms, and more preferably having 6 carbon atoms), a heteroaryl group (preferably having 5 to 18 carbon atoms, and more preferably having 5 and 6 carbon atoms), or a halogen atom (preferably a fluorine atom or a chlorine atom) is preferable.

In the present specification, unless otherwise specified, the above-described alkyl group is preferable as an alkyl group moiety in the alkoxy group. The alkyl group moiety in the alkylthio group is preferably the above-described alkyl group.

In the alkoxy group which may have a substituent, the substituent which can be contained in the alkoxy group includes the same examples as the substituent in the alkyl group which may have a substituent. In the alkylthio group which may have a substituent, the substituent which can be contained in the alkylthio group includes the same examples as the substituent in the alkyl group which may have a substituent.

In the present specification, the alkenyl group may be any of linear, branched, or cyclic, unless otherwise specified. The number of carbon atoms of the above-described alkenyl group is preferably 2 to 20. In the alkenyl group which may have a substituent, the substituent which can be contained in the alkenyl group includes the same examples as the substituent in the alkyl group which may have a substituent.

In the present specification, an alkynyl group may be any of linear, branched, or cyclic, unless otherwise specified. The number of carbon atoms of the above-described alkynyl group is preferably 2 to 20. In the alkynyl group which may have a substituent, the substituent which can be contained in the alkynyl group includes the same examples as the substituent in the alkyl group which may have a substituent.

In the present specification, an aromatic ring constituting the aromatic ring structure or the aromatic ring group may be any of a monocyclic ring or a polycyclic ring (for example, 2 to 6 rings or the like), unless otherwise specified. The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) aromatic ring structures being fused, as a ring structure.

The number of ring member atoms of the aromatic ring is preferably 5 to 15.

The aromatic ring may be any of an aromatic hydrocarbon ring or an aromatic heterocyclic ring.

In a case where the aromatic ring is an aromatic heterocyclic ring, the number of heteroatoms contained as ring member atoms is, for example, 1 to 10. Examples of the heteroatoms include a nitrogen atom, a sulfur atom, an oxygen atom, a selenium atom, a tellurium atom, a phosphorus atom, a silicon atom, and a boron atom.

Examples of the aromatic hydrocarbon ring include a benzene ring, a naphthalene ring, an anthracene ring, and a phenanthrene ring.

Examples of the aromatic heterocyclic ring include a pyridine ring, a pyrimidine ring, a pyridazine ring, a pyrazine ring, a triazine ring (for example, a 1,2,3-triazine ring, a 1,2,4-triazine ring, a 1,3,5-triazine ring, and the like), a tetrazine ring (for example, a 1,2,4,5-tetrazine ring and the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzimidazole ring, a benzoxazole ring, a benzothiazole ring, a naphthopyrrole ring, a naphthofuran ring, a naphthothiophene ring, a naphthimidazole ring, a naphthoxazole ring, a 3H-pyrrolidine ring, a pyrroloimidazole ring (for example, a 5H-pyrrolo[1,2-a]imidazole ring and the like), an imidazooxazole ring (for example, an imidazo[2,1-b]oxazole ring and the like), a thienothiazole ring (for example, a thieno[2,3-d]thiazole ring and the like), a benzothiadiazole ring, a benzodithiophene ring (for example, a benzo[1,2-b:4,5-b′]dithiophene ring and the like), a thienothiophene ring (for example, a thieno[3,2-b]thiophene ring and the like), a thiazolothiazole ring (for example, a thiazolo[5,4-d]thiazole ring and the like), a naphthodithiophene ring (for example, a naphtho[2,3-b:6,7-b′]dithiophene ring, a naphtho[2,1-b:6,5-b′]dithiophene ring, a naphtho[1,2-b:5,6-b′]dithiophene ring, a 1,8-dithiacyclopenta[b,g]naphthalene ring, and the like), a benzothienobenzothiophene ring, a dithieno[3,2-b:2′,3′-d]thiophene ring, and a 3,4,7,8-tetrathiadicyclopenta[a,e]pentalene ring.

In the aromatic ring which may have a substituent, examples of the type of the substituent which can be contained in the aromatic ring include a group exemplified by the substituent W. In a case where the aromatic ring has substituents, the number of substituents may be 1 or more (for example, 1 to 4 or the like).

In the present specification, the term “aromatic ring group” includes, for example, a group obtained by removing one or more hydrogen atoms (for example, 1 to 5 or the like) from the aromatic ring.

In the present specification, the term “aryl group” includes, for example, a group obtained by removing one hydrogen atom from a ring corresponding to an aromatic hydrocarbon ring among the above aromatic rings.

In the present specification, the term “heteroaryl group” includes, for example, a group obtained by removing one hydrogen atom from a ring corresponding to an aromatic heterocyclic ring among the above aromatic rings.

In the present specification, the term “arylene group” includes, for example, a group obtained by removing two hydrogen atoms from a ring corresponding to an aromatic hydrocarbon ring among the above aromatic rings.

In the present specification, the term “heteroarylene group” includes, for example, a group obtained by removing two hydrogen atoms from a ring corresponding to an aromatic heterocyclic ring among the above aromatic rings.

In an aromatic ring group which may have a substituent, an aryl group which may have a substituent, a heteroaryl group which may have a substituent, an arylene group which may have a substituent, and a heteroarylene group which may have a substituent, examples of a type of the substituents that these groups can have include a group exemplified by the substituent W. In a case where these groups each of which may have a substituent have substituents, the number of substituents may be 1 or more (for example, 1 to 4 or the like).

In the present specification, in a case where a plurality of identical symbols indicating a type or the number of groups are present in Formula, which indicates a chemical structure, contents of these plurality of identical symbols indicating a type or the number of groups are independent of each other, and the contents of the identical symbols may be the same or different from each other unless otherwise specified.

In the present specification, in a case where a plurality of identical groups (for example, alkyl groups and the like) are present in one Formula, which indicates a chemical structure, specific contents between these plurality of identical groups are independent of each other, and the specific contents between the plurality of identical groups may be the same or different from each other, unless otherwise specified.

The bonding direction of the divalent group (for example, —CO—O— and the like) denoted in the present specification, is not limited unless otherwise specified. For example, in a case where Y in a compound represented by a formula “X—Y—Z” is —CO—O—, the compound may be any of “X—O—CO—Z” or “X—CO—O—Z”.

### [Photoelectric Conversion Element]

Examples of the photoelectric conversion element according to the embodiment of the present invention include a first embodiment and a second embodiment.

The photoelectric conversion element according to the first embodiment is a photoelectric conversion element including in the following order, a conductive film, a photoelectric conversion film, and a transparent conductive film, in which the photoelectric conversion film contains a compound represented by Formula (1) (hereinafter, referred to as a “specific compound 1”).

The photoelectric conversion element according to the second embodiment is a photoelectric conversion element including in the following order, a conductive film, a photoelectric conversion film, and a transparent conductive film, in which the photoelectric conversion film contains a compound represented by Formula (2) (hereinafter, referred to as a “specific compound 2”).

Hereinafter, the specific compound 1 and the specific compound 2 are collectively referred to as a specific compound.

Examples of the feature point of the present invention include the point that the specific compound is contained, and it is presumed that the specific compound has a characteristic chemical structure, and thus the manufacturing suitability of the photoelectric conversion film containing the specific compound is excellent. In particular, it is considered that the above-described effect is exhibited by the point that the specific compound 1 has A¹ having a structure containing a nitrogen atom at a specific position, and the point that the specific compound 2 has A² having a structure containing a nitrogen atom at a specific position and D² having a specific structure.

Hereinafter, the fact that the manufacturing suitability is more excellent is also referred to as “effect of the present invention is more excellent”.

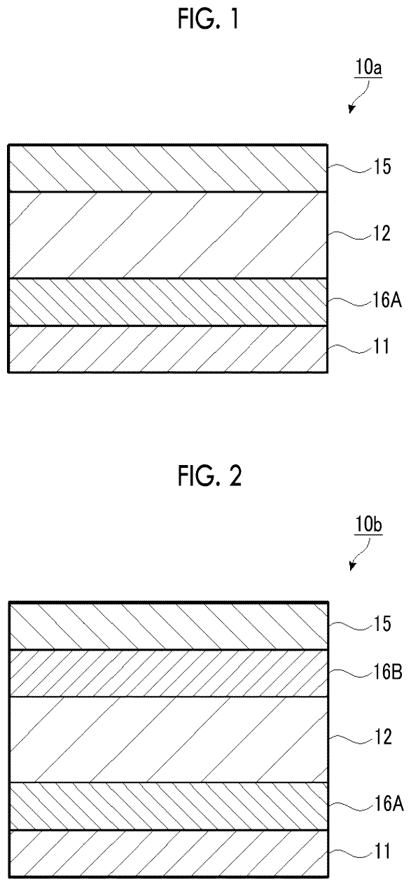

FIG. 1 is a schematic cross-sectional view of one embodiment of a photoelectric conversion element according to the embodiment of the present invention.

A photoelectric conversion element 10a illustrated in FIG. 1 has a configuration in which a conductive film (hereinafter, also referred to as a “lower electrode”) 11 functioning as a lower electrode, an electron blocking film 16A, a photoelectric conversion film 12 containing the specific compound, and a transparent conductive film (hereinafter, also referred to as an “upper electrode”) 15 functioning as an upper electrode are laminated in this order.

FIG. 2 illustrates a configuration example of another photoelectric conversion element. A photoelectric conversion element 10b illustrated in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of the performance and power consumption, the applied voltage is more preferably 1×10⁻⁴ to 1× 10⁻⁷ V/cm, and still more preferably 1×10⁻³ to 5×10⁻⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

In a case where a geometric isomer that can be distinguished based on a C═C double bond is present in the specific compound, the specific compound includes any geometric isomers. That is, both the cis isomer and the trans isomer, which are distinguished based on the C═C double bond, are included in the specific compound.

Hereinafter, the form of each layer constituting the photoelectric conversion element according to the first embodiment of the present invention will be described in detail.

### <<Photoelectric Conversion Element According to First Embodiment>>

The photoelectric conversion element according to the first embodiment is a photoelectric conversion element including, in the following order, a conductive film, a photoelectric conversion film, and a transparent conductive film, in which the photoelectric conversion film contains the specific compound 1.

### [Photoelectric Conversion Film]

The photoelectric conversion element according to the first embodiment has a photoelectric conversion film.

### <Specific Compound 1>

The photoelectric conversion film contains a specific compound 1.

$${D}^{1} = {A}^{1} \qquad ( 1 )$$

In Formula (1), A¹ represents a group represented by any of Formulae (A-1) to (A-3). D¹ represents a divalent organic group.

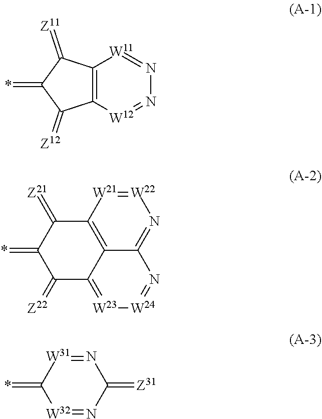

In Formula (A-1), * represents a bonding position. W¹¹ and W¹² each independently represent —CR^{W11}═ or a nitrogen atom. R^{W11} represents a hydrogen atom or a substituent. Z¹¹ and Z¹² each independently represent an oxygen atom, a sulfur atom, a selenium atom, ═NR^{Z11}, or ═C(R^{Z12})(R^{Z13}). R^{Z11} to R^{Z13} each independently represent a hydrogen atom or a substituent.

In Formula (A-2), * represents a bonding position. W²¹ to W²⁴ each independently represent —CR^{W21}═ or a nitrogen atom. R^{W21} represents a hydrogen atom or a substituent. Z²¹ and Z²² each independently represent an oxygen atom, a sulfur atom, a selenium atom, ═NR^{Z21}, or =C(R^{Z22})(R^{Z23}). R^{Z21} to R^{Z23} each independently represent a hydrogen atom or a substituent.

In Formula (A-3), * represents a bonding position. W³¹ and W³² each independently represent —CR^{W31}═ or a nitrogen atom. R^{W31} represents a hydrogen atom, a halogen atom, a cyano group, an aromatic ring group which may have a substituent, an aliphatic hydrocarbon group which may have a substituent, —OR^{W32}, —SR^{W33}, —Si(R^{W34})₃, —N(R^{W35})₂, or a group having a phosphorus atom. R^{W32} and R^{W33} each independently represent a substituent. R^{W34} and R^{W35} each independently represent a hydrogen atom or a substituent. Z³¹ represents an oxygen atom, a sulfur atom, a selenium atom, =NR^{Z31}, or =C(R^{Z32})(R^{Z33}). R^{Z31} to R^{Z33} each independently represent a hydrogen atom or a substituent.

In Formula (A-1), W¹¹ and W¹² each independently represent —CR^{W11}═ or a nitrogen atom. R^{W11} represents a hydrogen atom or a substituent.

It is preferable that at least one of W¹¹ or W¹² represents —CR^{W11}—, and it is more preferable that W¹¹ and W¹² represent —CR^{W11}═.

Examples of the above-described substituent include a group exemplified by the substituent W.

R^{W11} is preferably a hydrogen atom.

In a case where a plurality of R^{W11}'s are present, R^{W11}'s may be the same or different from each other.

In Formula (A-1), Z¹¹ and Z¹² each independently represent an oxygen atom, a sulfur atom, a selenium atom, =NR^{Z11}, or ═C(R^{Z12})(R^{Z13}). R^{Z11} to R^{Z13} each independently represent a hydrogen atom or a substituent.

Z¹¹ and Z¹² are preferably an oxygen atom or a sulfur atom, and more preferably an oxygen atom.

Examples of the substituent represented by R^{Z11} to R^{Z13} include a group exemplified by the substituent W.

In a case where a plurality of R^{Z11}'s are present, R^{Z11}'s may be the same or different from each other. In a case where a plurality of R^{Z12}'s are present, R^{Z12}'s may be the same or different from each other. In a case where a plurality of R^{Z13}'s are present, R^{Z13}'s may be the same or different from each other.

In Formula (A-2), W²¹ to W²⁴ each independently represent —CR^{W21}═ or a nitrogen atom. R^{W21} represents a hydrogen atom or a substituent.

It is preferable that at least one of W²¹, . . . , or W²⁴ represents —CR^{W21}═, it is more preferable that at least two of W²¹, . . . , or W²⁴ represent —CR^{W21}═, and it is still more preferable that W²¹ to W²⁴ represent —CR^{W21}═.

In addition, it is also preferable that W²² and W²⁴ represent —CR^{W21}═ and R^{W21} represents an alkyl group having a fluorine atom.

Examples of the substituent represented by R^{W21} include a group exemplified by the substituent W.

R^{W21} is preferably a hydrogen atom.

In a case where a plurality of R^{W21}'s are present, R^{W21}'s may be the same or different from each other.

In Formula (A-2), Z²¹ and Z²² each independently represent an oxygen atom, a sulfur atom, a selenium atom, =NR^{Z21}, or =C(R^{Z22})(R^{Z23}). R^{Z21} to R^{Z23} each independently represent a hydrogen atom or a substituent.

Z²¹ and Z²² are preferably an oxygen atom or a sulfur atom, and more preferably an oxygen atom.

Examples of R^{Z21} to R^{Z23} include the group represented by R^{Z11} to R^{Z13}, respectively. In a case where a plurality of R^{Z21}'s are present, R^{Z21}'s may be the same or different from each other. In a case where a plurality of R^{Z22}'s are present, R^{Z22}'s may be the same or different from each other. In a case where a plurality of R^{Z23}'s are present, R^{Z23}'s may be the same or different from each other.

In Formula (A-3), W³¹ and W³² each independently represent —CR^{W31}═ or a nitrogen atom. R^{W31} represents a hydrogen atom, a halogen atom, a cyano group, an aromatic ring group which may have a substituent, an aliphatic hydrocarbon group which may have a substituent, —OR^{W32}, —SRW³³, —Si(R^{W34})₃, —N(R^{W35})₂, or a group having a phosphorus atom. R^{W32} and R^{W33} each independently represent a substituent. R^{W34} and R^{W35} each independently represent a hydrogen atom or a substituent.

It is preferable that W³¹ and W³² represent —CR^{W31}═.

In addition, it is also preferable that W³¹ and W³² represent —CR^{W31}—, and R^{W31} represents an alkyl group having a fluorine atom.

The above-described aromatic ring group may be any of an aryl group which may have a substituent or a heteroaryl group which may have a substituent.

The above-described aliphatic hydrocarbon group may be any of linear, branched, or cyclic, and may be any of saturated or unsaturated.

Examples of the above-described aliphatic hydrocarbon group include an alkyl group, an alkenyl group, and an alkynyl group, which may have a substituent.

Examples of a substituent which can be included in the above-described aromatic ring group and the above-described aliphatic hydrocarbon group include a group exemplified by the substituent W.

Examples of the substituent represented by R^{W32} to R^{W35} include a group exemplified by the substituent W.

R^{W31} is preferably a hydrogen atom.

In a case where a plurality of R^{W31}'s are present, R^{W31}'s may be the same or different from each other.

In Formula (A-3), Z³¹ represents an oxygen atom, a sulfur atom, a selenium atom, =NR^{Z31}, or =C(R^{Z32})(R^{Z33}). R^{Z31} to R^{Z33} each independently represent a hydrogen atom or a substituent.

Z³¹ is preferably an oxygen atom or a sulfur atom and more preferably an oxygen atom.

Examples of R^{Z31} to R^{Z33} include the group represented by R^{Z11} to R^{Z13}, respectively.

In a case where a plurality of R^{Z31}'s are present, R^{Z31}'s may be the same or different from each other. In a case where a plurality of R^{Z32}'s are present, R^{Z32}'s may be the same or different from each other. In a case where a plurality of R^{Z33}'s are present, R^{Z33}'s may be the same or different from each other.

In Formulae (A-1) to (A-3), it is preferable that Z¹¹, Z¹², Z²¹, Z²², and Z³¹ are an oxygen atom or a sulfur atom.

In Formula (1), D¹ represents a divalent organic group.

The divalent organic group is a group that contains one or more carbon atoms and contains=*. * represents a bonding position with A¹ in Formula (1).

The above-described divalent organic group is not particularly limited as long as it is a group satisfying the above.

D¹ may include the group represented by any of Formulae (A-1) to (A-3) described above as a partial structure.

D¹ is preferably a group represented by Formula (D-1).

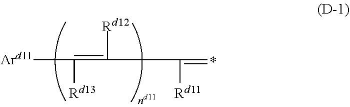

In Formula (D-1), * represents a bonding position. Ar^{d11} represents a substituent having an aromatic ring. R^{d11} to R^{d13} each independently represent a hydrogen atom or a substituent. n^{d11} represents an integer of 0 to 5.

In Formula (D-1), R^{d11} to R^{d13} each independently represent a hydrogen atom or a substituent.

Examples of the substituent represented by R^{d11} to R^{d13} include a group exemplified by the substituent W.

R^{d11} to R^{d13} are preferably a hydrogen atom.

In a case where a plurality of R^{d12}'s are present, R^{d12}'s may be the same or different from each other. In a case where a plurality of R^{d13}'s are present, R^{d13}'s may be the same or different from each other.

In Formula (D-1), n^{d11} represents an integer of 0 to 5.

n^{d11} is preferably 0 or 1, and more preferably 0.

In Formula (D-1), Ar^{d11} represents a substituent having an aromatic ring.

The substituent having an aromatic ring is a group having an aromatic ring in a part or all of the substituent. The substituent having an aromatic ring may include the group represented by any of Formulae (A-1) to (A-3) described above as a partial structure.

Ar^{d11} is preferably an aryl group which may have a substituent or a heteroaryl group which may have a substituent. It is also preferable that Ar^{d11} is a substituent having a fused polycyclic aromatic heterocyclic ring.

Examples of a substituent which can be included in the above-described aryl group and the above-described heteroaryl group include a group exemplified by the substituent W.

Ar^{d11} may be any of monocyclic ring or a polycyclic ring. The above-described polycyclic ring may be a fused ring.

The total number of ring members in the aromatic ring included in Ar^{d11} is preferably 5 to 40, more preferably 10 to 30, and still more preferably 20 to 30.

The aromatic ring may be any of an aromatic hydrocarbon ring or an aromatic heterocyclic ring.

Examples of the aromatic hydrocarbon ring include a benzene ring, a naphthalene ring, an anthracene ring, a phenanthrene ring, and a ring obtained by combining these rings.

Examples of the aromatic heterocyclic ring include a thiophene ring, a furan ring, a pyran ring, a thiazole ring, a pyrrole ring, a pyridine ring, a pyrazine ring, a pyrimidine ring, a pyridazine ring, an oxazole ring, a selenophene ring, an imidazole ring, a quinoxaline ring, a benzothiazole ring, and a ring obtained by combining these rings.

Ar^{d11} may further have another ring in addition to the above-described aromatic ring. The other ring may be fused to the above-described aromatic ring to form a fused ring.

Examples of the above-described other ring include a cycloalkane ring, a piperidine ring, a piperazine ring, an imidazolidine ring, and a ring obtained by combining these rings.

Hereinafter, a bonding mode of each group will be described with specific examples of the specific compound.

For example, in Formula (1), in a case where D¹ is a group represented by Formula (D-1) and A¹ is a group represented by Formula (A-1), the specific compound 1 is Compound DA1. In a case where D¹ is the group represented by Formula (D-1) and A¹ is a group represented by Formula (A-2), the specific compound 1 is Compound DA2. In a case where D¹ is the group represented by Formula (D-1) and A¹ is a group represented by Formula (A-3), the specific compound 1 is Compound DA3.

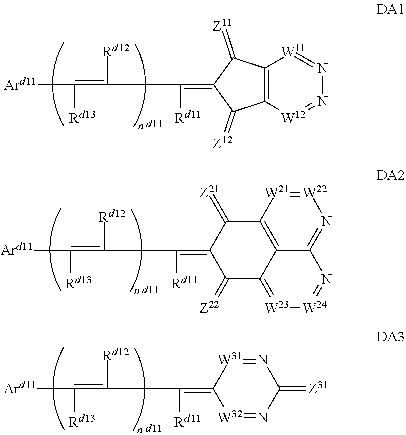

Ar^{d11} is preferably a group represented by any of Formulae (Ar-1) to (Ar-9), more preferably a group represented by any of Formulae (Ar-1) to (Ar-3), (Ar-8), or (Ar-9), still more preferably a group represented by Formula (Ar-1), particularly preferably a group represented by Formula (Ar-10), and most preferably a group represented by Formula (Ar-11).

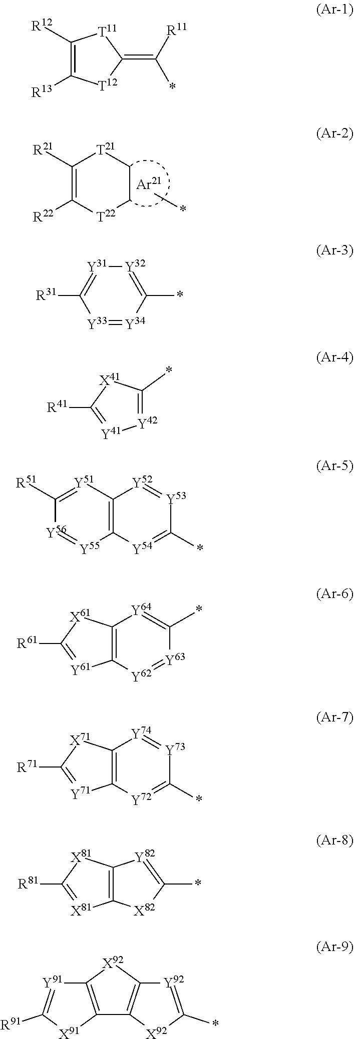

In Formula (Ar-1), * represents a bonding position. R¹¹ to R¹³ each independently represent a hydrogen atom or a substituent. At least two of R¹¹, R¹², or R¹³ may be bonded to each other to form a ring. T¹¹ and T¹² each independently represent an oxygen atom, a sulfur atom, a selenium atom, —NR¹⁴—, or —C(R¹⁵)(R¹⁶)—. R¹⁴ to R¹⁶ each independently represent a hydrogen atom, an alkyl group which may have a substituent, or an aromatic ring group which may have a substituent.

In Formula (Ar-2), * represents a bonding position. Ar²¹ represents an aromatic ring which includes 2 or more carbon atoms and may have a substituent. R²¹ and R²² each independently represent a hydrogen atom or a substituent. R²¹ and R²² may be bonded to each other to form a ring. T²¹ and T²² each independently represent an oxygen atom, a sulfur atom, a selenium atom, —C(R²³)(R²⁴)—, —Si(R²⁵)(R²⁶)—, —NR²⁷—, or >C═R²⁸. R²³ to R²⁷ each independently represent a hydrogen atom or a substituent. R²³ and R²⁴, or R²⁵ and R²⁶ may be bonded to each other to form a ring. R²⁸ represents an oxygen atom, a sulfur atom, or =C(R²⁹)(R³⁰). R²⁹ and R³⁰ each independently represent a hydrogen atom or a substituent. At least one of R²⁹ or R³⁰ and at least one of Ar²¹, R²¹, or R²² may be bonded to each other to form a ring.

In Formula (Ar-3), * represents a bonding position. R³¹ represents a hydrogen atom or a substituent. Y³¹ to Y³⁴ each independently represent —CR³²═ or a nitrogen atom. R³² represents a hydrogen atom, a halogen atom, a trifluoromethyl group, or a cyano group. In a case where at least two of Y³¹, . . . , or Y³⁴ are —CR³²═, R³²'s may be bonded to each other to form a ring.

In Formula (Ar-4), * represents a bonding position. X⁴¹ represents an oxygen atom, a sulfur atom, a selenium atom, or —NR⁴²—. R⁴¹ and R⁴² each independently represent a hydrogen atom or a substituent. Y⁴¹ and Y⁴² each independently represent —CR⁴³═ or a nitrogen atom. R⁴³ represents a hydrogen atom, a halogen atom, a trifluoromethyl group, or a cyano group. In a case where Y⁴¹ and Y⁴² are —CR⁴³═, R⁴³'s may be bonded to each other to form a ring.

In Formula (Ar-5), * represents a bonding position. R⁵¹ represents a hydrogen atom or a substituent. Y⁵¹ to Y⁵⁶ each independently represent —CR⁵²═ or a nitrogen atom. R⁵² represents a hydrogen atom, a halogen atom, a trifluoromethyl group, or a cyano group. In a case where at least two of Y⁵¹, . . . , or Y⁵⁶ are —CR⁵²═, R⁵²'s may be bonded to each other to form a ring.

In Formula (Ar-6), * represents a bonding position. X⁶¹ represents an oxygen atom, a sulfur atom, a selenium atom, or —NR⁶²—. R⁶¹ and R⁶² each independently represent a hydrogen atom or a substituent. Y⁶¹ to Y⁶⁴ each independently represent —CR⁶³═ or a nitrogen atom. R⁶³ represents a hydrogen atom, a halogen atom, a trifluoromethyl group, or a cyano group. In a case where at least two of Y⁶¹, . . . , or Y⁶⁴ are —CR⁶³═, R⁶³'s may be bonded to each other to form a ring.

In Formula (Ar-7), * represents a bonding position. X⁷¹ represents an oxygen atom, a sulfur atom, a selenium atom, or —NR⁷²—. R⁷¹ and R⁷² each independently represent a hydrogen atom or a substituent. Y⁷¹ to Y⁷⁴ each independently represent —CR⁷³═ or a nitrogen atom. R⁷³ represents a hydrogen atom, a halogen atom, a trifluoromethyl group, or a cyano group. In a case where at least two of Y⁷¹, . . . , or Y⁷⁴ are —CR⁷³═, R⁷³'s may be bonded to each other to form a ring.

In Formula (Ar-8), * represents a bonding position. X⁸¹ and X⁸² each independently represent an oxygen atom, a sulfur atom, a selenium atom, or —NR⁸²—. R⁸¹ and R⁸² each independently represent a hydrogen atom or a substituent. Y⁸¹ and Y⁸² each independently represent —CR⁸³═ or a nitrogen atom. R⁸³ represents a hydrogen atom, a halogen atom, a trifluoromethyl group, or a cyano group.

In Formula (Ar-9), * represents a bonding position. X⁹¹ to X⁹³ each independently represent an oxygen atom, a sulfur atom, a selenium atom, or —NR⁹²—. R⁹¹ and R⁹² each independently represent a hydrogen atom or a substituent. Y⁹¹ and Y⁹² each independently represent —CR⁹³═ or a nitrogen atom. R⁹³ represents a hydrogen atom, a halogen atom, a trifluoromethyl group, or a cyano group.

In Formula (Ar-1), R¹¹ to R¹³ each independently represent a hydrogen atom or a substituent. At least two of R¹¹, R¹², or R¹³ may be bonded to each other to form a ring.

Examples of the substituent represented by R¹¹ include a group exemplified by the substituent W.

R¹¹ is preferably a hydrogen atom.

Examples of R¹² and R¹³ include the group exemplified by the substituent W, and an alkyl group which may have a substituent or an aromatic ring group which may have a substituent (preferably, an aryl group which may have a substituent) is preferable. In addition, R¹² and R¹³ are also preferably a group represented by *=A¹. A¹ has the same meaning as A¹ in Formula (1).

R¹² and R¹³ are preferably bonded to each other to form a ring. The above-described ring formed is preferably an aromatic heterocyclic ring and more preferably a quinoxaline ring or a pyrazine ring. In addition, the above-described ring formed is also preferably an aromatic hydrocarbon ring and more preferably a benzene ring. The above-described ring formed may further have a substituent. Examples of the above-described substituent include a group exemplified by the substituent W, and an alkyl group which may have a substituent, a chlorine atom, a fluorine atom, or a cyano group is preferable, and an alkyl group or a chlorine atom is more preferable.

In Formula (Ar-1), T¹¹ and T¹² each independently represent an oxygen atom, a sulfur atom, a selenium atom, —NR¹⁴—, or —C(R¹⁵)(R¹⁶)—. R¹⁴ to R¹⁶ each independently represent a hydrogen atom, an alkyl group which may have a substituent, or an aromatic ring group which may have a substituent.

T¹¹ and T¹² are preferably —NR¹⁴— or —C(R¹⁵)(R¹⁶)—.

In a case where at least one of T¹¹ or T¹² is —C(R¹⁵)(R¹⁶)—, R¹⁵ and R¹⁶ may be bonded to each other to form a ring. The above-described ring formed is preferably a cycloalkane ring and more preferably a cyclohexane ring.

The above-described alkyl group may be any of linear, branched, or cyclic.

The number of carbon atoms of the above-described alkyl group is preferably 1 to 10 and more preferably 1 to 3.

The above-described aromatic ring group may be any of an aryl group which may have a substituent or a heteroaryl group which may have a substituent, and is preferably an aryl group which may have a substituent.

The aromatic ring group may be any of a monocyclic ring or a polycyclic ring. The above-described polycyclic ring may be a fused ring.

The number of carbon atoms of the above-described aromatic ring group is preferably 3 to 30 and more preferably 3 to 15.

The number of substituents included in the above-described aromatic ring group is preferably 1 to 5 and more preferably 2 or 3.

Examples of a substituent which can be included in the above-described alkyl group and the above-described aromatic ring group include a group exemplified by the substituent W. The substituent which can be included in the above-described aromatic ring group is preferably an alkyl group or a heteroaryl group and more preferably an alkyl group having 1 to 3 carbon atoms.

The above-described aromatic ring group is preferably a phenyl group, a naphthyl group, or a fluorenyl group, which may have a substituent, more preferably a phenyl group which may have a substituent, and still more preferably a phenyl group having a substituent.

It is also preferable that T¹¹ and T¹² represent the same group.

In a case where a plurality of R¹⁴'s are present, R¹⁴'s may be the same or different from each other. In a case where a plurality of R¹⁵'s are present, R¹⁵'s may be the same or different from each other. In a case where a plurality of R¹⁶'s are present, R¹⁶'s may be the same or different from each other.

R¹⁴ to R¹⁶ may be a group represented by Formula (R-1).

R¹⁴ is preferably a group represented by Formula (R-1).

R¹⁵ and R¹⁶ are preferably an alkyl group which may have a substituent and more preferably an unsubstituted alkyl group.

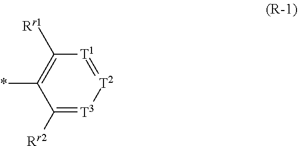

In Formula (R-1), * represents a bonding position. R^{r1} and R^{r2} each independently represent an alkyl group which may have a substituent or an aromatic ring group which may have a substituent.

T¹ to T³ each independently represent —CR^{r3}═ or a nitrogen atom. R^{r3} represents a hydrogen atom or a substituent.

In Formula (R-1), R^{r1} and R^{r2} each independently represent an alkyl group which may have a substituent or an aromatic ring group which may have a substituent.

Examples of the above-described alkyl group and the above-described aromatic ring group include the alkyl group which may have a substituent and the aromatic ring group which may have a substituent, which are represented by R¹⁴ to R¹⁶

R^{r1} and R^{r2} are preferably an alkyl group which may have a substituent or an aryl group which may have a substituent.

In Formula (R-1), T¹ to T³ each independently represent —CR^{r3}═ or a nitrogen atom. R¹³ represents a hydrogen atom or a substituent.

It is preferable that T¹ and T³ represent —CH=, and T² represents —CR¹³═.

Examples of the above-described substituent include a group exemplified by the substituent W.

R^{r3} is preferably a hydrogen atom or an alkyl group.

In a case where a plurality of R^{r3}'s are present, R^{r3}'s may be the same or different from each other.

In Formula (Ar-2), Ar²¹ represents an aromatic ring which includes 2 or more carbon atoms and may have a substituent.

The “2 or more carbon atoms” means that the aromatic ring represented by Ar²¹ includes two carbon atoms constituting a bonding portion between the aromatic ring represented by Ar²¹ and the ring including T²¹ and T²², and may further include a carbon atom other than the two carbon atoms. In other words, the aromatic ring represented by Ar²¹ includes the above-described two carbon atoms as ring member atoms.

The aromatic ring group may be any of a monocyclic ring or a polycyclic ring. The above-described polycyclic ring may be a fused ring.

The number of ring members in the above-described aromatic ring is preferably 3 to 12 and more preferably 3 to 6.

The number of carbon atoms of the above-described aromatic ring is 2 or more, preferably 3 to 20, and more preferably 5 to 12.

The aromatic ring may be any of an aromatic hydrocarbon ring or an aromatic heterocyclic ring.

Examples of the aromatic hydrocarbon ring include a benzene ring, a naphthalene ring, an anthracene ring, a phenanthrene ring, and a ring obtained by combining these rings.

Examples of the aromatic heterocyclic ring include a thiophene ring, a furan ring, a pyran ring, a thiazole ring, a pyrrole ring, a pyridine ring, a pyrazine ring, a pyrimidine ring, a pyridazine ring, an oxazole ring, a selenophene ring, an imidazole ring, a quinoxaline ring, a benzothiazole ring, and a ring obtained by combining these rings.

The above-described aromatic ring is more preferably a benzene ring, a naphthalene ring, or a thiophene ring.

Examples of the substituent which can be contained in the above-described aromatic ring include a group exemplified by the substituent W.

In Formula (Ar-2), R²¹ and R²² each independently represent a hydrogen atom or a substituent. R²¹ and R²² may be bonded to each other to form a ring.

Examples of the substituent represented by R²¹ and R²² include the substituent represented by R¹² and R¹³.

Examples of the ring formed by the bonding of R²¹ and R²² to each other include a ring formed by the bonding of R¹² and R¹³ to each other, and an aromatic hydrocarbon ring is preferable and a benzene ring is more preferable.

In Formula (Ar-2), T²¹ and T²² each independently represent an oxygen atom, a sulfur atom, a selenium atom, —C(R²³)(R²⁴)—, —Si(R²⁵)(R²⁶)—, —NR²⁷—, or >C═R²⁸. R²³ to R²⁷ each independently represent a hydrogen atom or a substituent. R²³ and R²⁴, or R²⁵ and R²⁶ may be bonded to each other to form a ring. R²⁸ represents an oxygen atom, a sulfur atom, or ═C(R²⁹)(R³⁰). R²⁹ and R³⁰ each independently represent a hydrogen atom or a substituent. At least one of R²⁹ or R³⁰ and at least one of Ar²¹, R²¹, or R²² may be bonded to each other to form a ring.

T²¹ and T²² are preferably —C(R²³)(R²⁴)— or —NR²⁷—.

Examples of the substituent represented by R²³ to R²⁷ include a group exemplified by the substituent W, and an alkyl group is preferable and an alkyl group having 1 to 3 carbon atoms is more preferable. In addition, the substituent represented by R²⁷ is also preferably an aromatic ring group and more preferably a benzene ring group.

Examples of the substituent represented by R²⁹ and R³⁰ include a group exemplified by the substituent W.

At least one of R²⁹ or R³⁰ and at least one of Ar²¹, R²¹, or R²² may be bonded to each other to form a ring.

In a case where a plurality of the same notations are present, the same notations may be the same or different from each other.

In Formula (Ar-3), R³¹ represents a hydrogen atom or a substituent.

Examples of the substituent represented by R³¹ include a diarylamino group and the substituent represented by R¹² and R¹³, and an aryl group which may have a substituent or a heteroaryl group which may have a substituent is preferable. The substituent which can be included in the above-described aryl group and the above-described heteroaryl group is preferably a -aromatic heterocyclic ring-aromatic hydrocarbon ring-1,3-dicarbonyl ring group.

In Formula (Ar-3), Y³¹ to Y³⁴ each independently represent —CR³²═ or a nitrogen atom. R³² represents a hydrogen atom, a halogen atom, a trifluoromethyl group, or a cyano group. In a case where at least two of Y³¹, . . . , or Y³⁴ are —CR³²═, R³²'s may be bonded to each other to form a ring.

It is preferable that at least two of Y³¹, . . . , or Y³⁴ represent —CR³²═, and it is more preferable that Y³¹ to Y³⁴ represent —CR³²═.

R³² is preferably a hydrogen atom.

In a case where a plurality of R³²'s are present, R³²'s may be the same or different from each other.

In Formula (Ar-4), X⁴¹ represents an oxygen atom, a sulfur atom, a selenium atom, or —NR⁴²—.

X⁴¹ is preferably an oxygen atom or a sulfur atom and more preferably a sulfur atom.

In Formula (Ar-4), R⁴¹ and R⁴² each independently represent a hydrogen atom or a substituent.

Examples of the substituent represented by R⁴¹ and R⁴² include the substituent represented by R¹² and R¹³, and an aryl group which may have a substituent or a heteroaryl group which may have a substituent is preferable.

In a case where a plurality of R⁴²'s are present, R⁴²'s may be the same or different from each other.

In Formula (Ar-4), Y⁴¹ and Y⁴² each independently represent —CR⁴³═ or a nitrogen atom. R⁴³ represents a hydrogen atom, a halogen atom, a trifluoromethyl group, or a cyano group. In a case where Y⁴¹ and Y⁴² are —CR⁴³═, R⁴³'s may be bonded to each other to form a ring.

At least one of Y⁴¹ or Y⁴² preferably represents —CR⁴³═.

R⁴³ is preferably a hydrogen atom.

In a case where a plurality of R⁴³'s are present, R⁴³'s may be the same or different from each other.

In Formula (Ar-5), R⁵¹ represents a hydrogen atom or a substituent.

Examples of the substituent represented by R⁵¹ include the substituent represented by R¹² and R¹³.

In Formula (Ar-5), Y⁵¹ to Y⁵⁶ each independently represent —CR⁵²═ or a nitrogen atom. R⁵² represents a hydrogen atom, a halogen atom, a trifluoromethyl group, or a cyano group. In a case where at least two of Y⁵¹, . . . , or Y⁵⁶ are —CR⁵²═, R⁵²'s may be bonded to each other to form a ring.

It is preferable that at least two of Y⁵¹, . . . , or Y⁵⁶ represent —CR⁵²═, and it is more preferable that at least four of Y⁵¹, . . . , or Y⁵⁶ represent —CR⁵²═.

R⁵² is preferably a hydrogen atom.

In a case where a plurality of R⁵²'s are present, R⁵²'s may be the same or different from each other.

In Formula (Ar-6), X⁶¹ represents an oxygen atom, a sulfur atom, a selenium atom, or —NR⁶²—.

X⁶¹ is preferably an oxygen atom or a sulfur atom.

In Formula (Ar-6), R⁶¹ and R⁶² each independently represent a hydrogen atom or a substituent.

Examples of the substituent represented by R⁶¹ and R⁶² include the substituent represented by R¹² and R¹³.

In a case where a plurality of R⁶²'s are present, R⁶²'s may be the same or different from each other.

In Formula (Ar-6), Y⁶¹ to Y⁶⁴ each independently represent —CR⁶³═ or a nitrogen atom. R⁶³ represents a hydrogen atom, a halogen atom, a trifluoromethyl group, or a cyano group. In a case where at least two of Y⁶¹, . . . , or Y⁶⁴ are —CR⁶³═, R⁶³'s may be bonded to each other to form a ring.

It is preferable that at least one of Y⁶¹, . . . , or Y⁶⁴ represents —CR⁶³═, and it is more preferable that at least two of Y⁶¹, . . . , or Y⁶⁴ represent —CR⁶³═.

R⁶³ is preferably a hydrogen atom.

In a case where a plurality of R⁶³'s are present, R⁶³'s may be the same or different from each other.

In Formula (Ar-7), X⁷¹ represents an oxygen atom, a sulfur atom, a selenium atom, or —NR⁷²—.

X⁷¹ is preferably an oxygen atom or a sulfur atom.

In Formula (Ar-7), R⁷¹ and R⁷² each independently represent a hydrogen atom or a substituent.

Examples of the substituent represented by R⁷¹ and R⁷² include the substituent represented by R¹² and R¹³.

In a case where a plurality of R⁷²'s are present, R⁷²'s may be the same or different from each other.

In Formula (Ar-7), Y⁷¹ to Y⁷⁴ each independently represent —CR⁷³═ or a nitrogen atom. R⁷³ represents a hydrogen atom, a halogen atom, a trifluoromethyl group, or a cyano group. In a case where at least two of Y⁷¹, . . . , or Y⁷⁴ are —CR⁷³═, —CR⁷³═'s may be bonded to each other to form a ring.

It is preferable that at least one of Y⁷¹, . . . , or Y⁷⁴ represents —CR⁷³═, and it is more preferable that at least two of Y⁷¹, . . . , or Y⁷⁴ represent —CR⁷³═.

R⁷³ is preferably a hydrogen atom.

In a case where a plurality of R⁷³'s are present, R⁷³'s may be the same or different from each other.

In Formula (Ar-8), X⁸¹ and X⁸² each independently represent an oxygen atom, a sulfur atom, a selenium atom, or —NR⁸¹—.

It is preferable that one of X⁸¹ or X⁸² represents an oxygen atom or a sulfur atom, and the other represents —NR⁸¹—.

In a case where a plurality of R⁸²'s are present, R⁸²'s may be the same or different from each other.

In Formula (Ar-8), R⁸¹ and R⁸² represent a hydrogen atom or a substituent.

Examples of the substituent represented by R⁸¹ and R⁸² include the substituent represented by R¹² and R¹³, and an alkyl group which may have a substituent or an aromatic ring group which may have a substituent is preferable.

In Formula (Ar-8), Y⁸¹ and Y⁸² each independently represent —CR⁸³═ or a nitrogen atom. R⁸³ represents a hydrogen atom, a halogen atom, a trifluoromethyl group, or a cyano group.

At least one of X⁸¹ or X⁸² preferably represents —CR⁸³═.

R⁸³ is preferably a hydrogen atom.

In a case where a plurality of R⁸³'s are present, R⁸³'s may be the same or different from each other.

In Formula (Ar-9), X⁹¹ to X⁹³ each independently represent an oxygen atom, a sulfur atom, a selenium atom, or —NR⁹²—.

It is preferable that at least one of X⁹¹, X⁹², or X⁹³ represents an oxygen atom or a sulfur atom, and it is more preferable that at least two of X⁹¹, X⁹², or X⁹³ represent a sulfur atom.

In Formula (Ar-9), R⁹¹ and R⁹² each independently represent a hydrogen atom or a substituent.

Examples of the substituent represented by R⁹¹ and R⁹² include the substituent represented by R¹² and R¹³, and an alkyl group which may have a substituent or an aromatic ring group which may have a substituent is preferable.

The substituent represented by R⁹¹ is preferably a group represented by *=A¹ or a 1,3-dicarbonyl ring group. Examples of the 1,3-dicarbonyl ring group include a 1,3-indandione ring group, a 1,3-cyclohexanedione ring group, a 5,5-dimethyl-1,3-cyclohexanedione ring group, and a 1,3-dioxane-4,6-dione ring group.

In a case where a plurality of R⁹²'s are present, R⁹²'s may be the same or different from each other.

In Formula (Ar-9), Y⁹¹ and Y⁹² each independently represent —CR⁹³═ or a nitrogen atom. R⁹³ represents a hydrogen atom, a halogen atom, a trifluoromethyl group, or a cyano group.

At least one of Y⁹¹ or Y⁹² preferably represents —CR⁹³═

R⁹³ is preferably a hydrogen atom.

In a case where a plurality of R⁹³'s are present, R⁹³'s may be the same or different from each other.

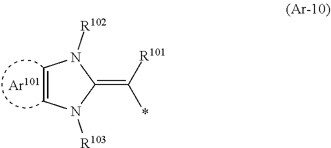

In Formula (Ar-10), * represents a bonding position. R¹⁰¹ represents a hydrogen atom or a substituent. R¹⁰² and R¹⁰³ each independently represent a hydrogen atom, an alkyl group which may have a substituent, or an aromatic ring group which may have a substituent. Ar¹⁰¹ represents an aromatic ring which includes 2 or more carbon atoms and may have a substituent.

In Formula (Ar-10), R¹⁰¹ represents a hydrogen atom or a substituent.

Examples of the substituent represented by R¹⁰¹ include the substituent represented by R¹¹.

R¹⁰¹ is preferably a hydrogen atom.

In Formula (Ar-10), R¹⁰² and R¹⁰³ each independently represent a hydrogen atom, an alkyl group which may have a substituent, or an aromatic ring group which may have a substituent.

Examples of R¹⁰² and R¹⁰³ include the group represented by R¹⁴.

In Formula (Ar-10), Ar¹⁰¹ represents an aromatic ring which includes 2 or more carbon atoms and may have a substituent.

The “2 or more carbon atoms” means that the aromatic ring represented by Ar¹⁰¹ includes two carbon atoms constituting a bonding portion between the aromatic ring represented by Ar¹⁰¹ and the ring including —NR¹⁰²— and —NR¹⁰³—, and may further include a carbon atom other than the two carbon atoms. In other words, the aromatic ring represented by Ar¹⁰¹ includes the above-described two carbon atoms as ring member atoms.

The aromatic ring group may be any of a monocyclic ring or a polycyclic ring. The above-described polycyclic ring may be a fused ring.

The number of ring members in the above-described aromatic ring is preferably 3 to 12.

The number of carbon atoms of the above-described aromatic ring is 2 or more, preferably 3 to 20, and more preferably 5 to 12.

The aromatic ring may be any of an aromatic hydrocarbon ring or an aromatic heterocyclic ring.

Examples of the aromatic hydrocarbon ring include a benzene ring, a naphthalene ring, an anthracene ring, a phenanthrene ring, and a ring obtained by combining these rings.

Examples of the aromatic heterocyclic ring include a thiophene ring, a furan ring, a pyran ring, a thiazole ring, a pyrrole ring, a pyridine ring, a pyrazine ring, a pyrimidine ring, a pyridazine ring, an oxazole ring, a selenophene ring, an imidazole ring, a quinoxaline ring, a benzothiazole ring, and a ring obtained by combining these rings.

The above-described aromatic ring is preferably an aromatic heterocyclic ring, and more preferably a quinoxaline ring or a pyrazine ring.

Examples of the substituent which can be included in the aromatic ring represented by Ar¹⁰¹ include a group exemplified by the substituent W, and an alkyl group which may have a substituent, a chlorine atom, a fluorine atom, or a cyano group is preferable.

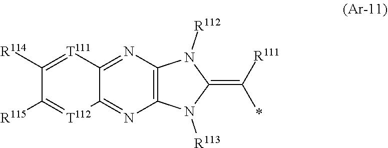

In Formula (Ar-11), * represents a bonding position. R¹¹¹ represents a hydrogen atom or a substituent. R¹¹² and R¹¹³ each independently represent a hydrogen atom, an alkyl group which may have a substituent, or an aromatic ring group which may have a substituent. R¹¹⁴ and R¹¹⁵ each independently represent a hydrogen atom, a halogen atom or an alkyl group which may have a substituent. T¹¹¹ and T¹¹² each independently represent a nitrogen atom or —CR¹¹⁶═. R¹¹⁶ represents a hydrogen atom, an alkyl group which may have a substituent, or an aromatic ring group which may have a substituent.

In Formula (Ar-11), R¹¹¹ represents a hydrogen atom or a substituent.

Examples of the substituent represented by R¹¹¹ include the substituent represented by R¹¹.

In Formula (Ar-11), R¹¹² and R¹¹³ each independently represent a hydrogen atom, an alkyl group which may have a substituent, or an aromatic ring group which may have a substituent.

Examples of R¹¹² and R¹¹³ include the group represented by R¹⁴.

In Formula (Ar-11), R¹¹⁴ and R¹¹⁵ each independently represent a hydrogen atom, a halogen atom, or an alkyl group which may have a substituent.

Examples of the above-described halogen atom include a fluorine atom, a chlorine atom, a bromine atom, and an iodine atom, and a fluorine atom or a chlorine atom is preferable.

The above-described alkyl group may be any of linear, branched, or cyclic.

The number of carbon atoms of the above-described alkyl group is preferably 1 to 10, more preferably 1 to 3, and still more preferably 1.

Examples of the substituent which can be contained in the above-described alkyl group include a group exemplified by the substituent W.

R¹¹⁴ and R¹¹⁵ is preferably an alkyl group which may have a substituent and more preferably an alkyl group having no substituent (unsubstituted alkyl group).

In Formula (Ar-11), T¹¹¹ and T¹¹² each independently represent a nitrogen atom or —CR¹¹⁶═. R¹¹⁶ represents a hydrogen atom, an alkyl group which may have a substituent, or an aromatic ring group which may have a substituent.

T¹¹¹ and T¹¹² are preferably-CR¹¹⁶═.

Examples of the alkyl group which may have a substituent and the aromatic ring group which may have a substituent, which is represented by R¹¹⁶, include the alkyl group which may have a substituent and the aromatic ring group which may have a substituent, which are represented by R¹⁴ to R¹⁶.

R¹¹⁶ is preferably a hydrogen atom.

In a case where a plurality of R¹¹⁶'s are present, R¹¹⁶'s may be the same or different from each other.

Hereinafter, a bonding mode of each group will be described with specific examples of the specific compound.

For example, in a case where, in Formula (1), D¹ is a group represented by Formula (D-1), n^{d11} is 0 and Ar^{d11} is a group represented by Formula (Ar-1) in Formula (D-1), and A¹ is Formula (A-1), the compound represented by Formula (1) is a compound represented by Formula (X-1). In addition, in a case where, in Formula (1), D¹ is the group represented by Formula (D-1), n^{d11} is 1 and Ar^{d11} is the group represented by Formula (Ar-3) in Formula (D-1), and A¹ is Formula (A-2), the compound represented by Formula (1) is a compound represented by Formula (X-2).

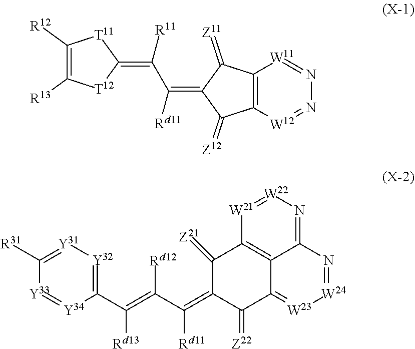

Examples of the specific compound 1 include the following compounds.

Compound 1-1 to Compound 1-7 are compounds in which Ar^{d11} is a group represented by Formula (Ar-1), Compound 1-8 is a compound in which Ar^{d11} is a group represented by Formula (Ar-3), Compound 1-9 and Compound 1-10 are compounds in which Ar^{d11} is a group represented by Formula (Ar-2), Compound 1-11 and Compound 1-12 are compounds in which Ar^{d11} is a group represented by Formula (Ar-8), Compound 1-13 and Compound 1-14 are compounds in which Ar^{d11} is a group represented by Formula (Ar-9), Compound 1-15 is a compound in which Ar^{d11} is a group represented by Formula (Ar-4), and Compound 1-16 is a compound in which Ar^{d11} is a group represented by Formula (Ar-3).

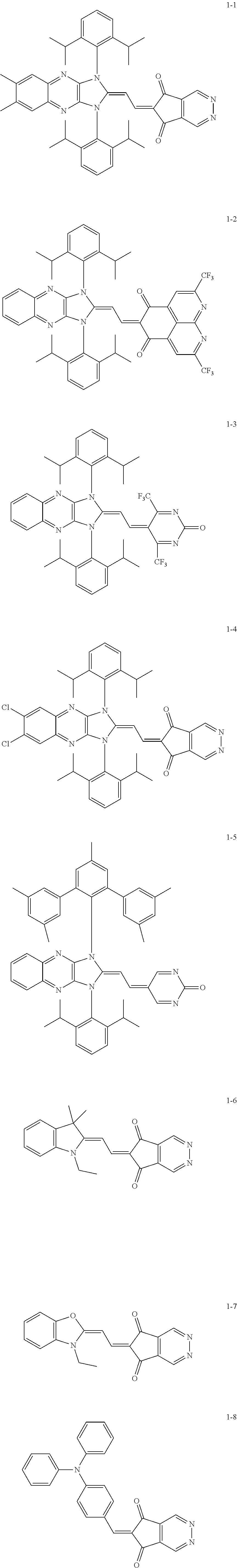

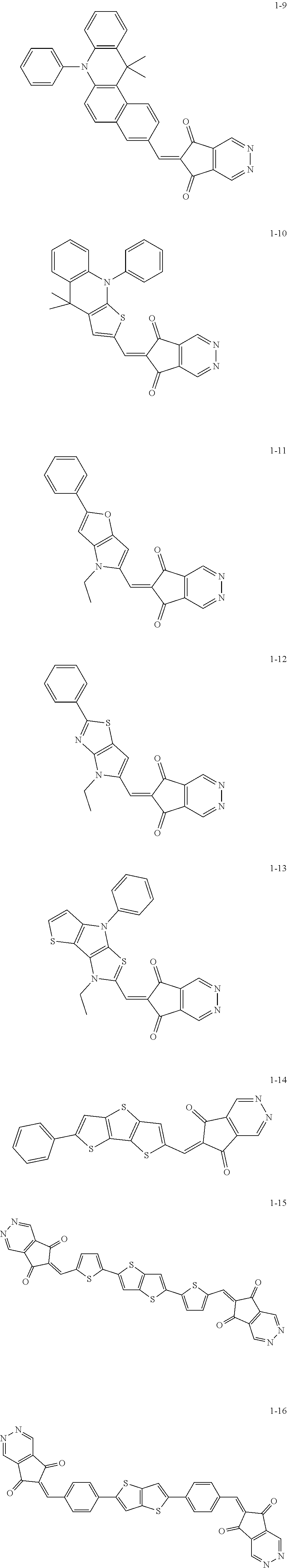

A molecular weight of the specific compound 1 is preferably 400 to 1,200, more preferably 400 to 1,000, and still more preferably 400 to 800.

In a case where the molecular weight is in the above-described range, it is presumed that the sublimation temperature of the specific compound 1 becomes low, and the photoelectric conversion efficiency is excellent also in a case where a photoelectric conversion film is formed at a high speed.

The specific compound 1 is particularly useful as a material of the photoelectric conversion film used for the imaging element, the optical sensor, or a photoelectric cell. The specific compound 1 often functions as a coloring agent in the photoelectric conversion film. The specific compound 1 can also be used as a coloring material, a liquid crystal material, an organic semiconductor material, a charge transport material, a pharmaceutical material, and a fluorescent diagnostic material.

In the specific compound 1, an ionization potential in a single film is preferably −6.0 to −5.0 eV from the viewpoints of stability in a case of using the compound as the p-type organic semiconductor and matching of energy levels between the compound and the n-type organic semiconductor.

The maximal absorption wavelength of the specific compound 1 is preferably in a wavelength range of 400 to 600 nm, and more preferably in a wavelength range of 450 to 580 nm.

The maximal absorption wavelength is a value measured in a solution state (solvent: chloroform) by an absorption spectrum of the specific compound 1 being adjusted to a concentration having an absorbance of about 0.5 to 1.0. Provided that in a case where the specific compound 1 is not soluble in chloroform, a value measured by using the specific compound 1 in which the specific compound 1 is vapor-deposited and formed into a film state is defined as a maximal absorption wavelength of the specific compound 1.

The specific compound 1 may be purified as necessary.

Examples of a purification method of the specific compound 1 include sublimation purification, purification using silica gel column chromatography, purification using gel permeation chromatography, reslurry washing, repurification by reprecipitation, purification using an adsorbent such as activated carbon, and recrystallization purification.

The specific compound 1 may be used alone or in combination of two or more types thereof.

A content of the specific compound 1 in the photoelectric conversion film (=film thickness of specific compound 1 in terms of single layer/film thickness of photoelectric conversion film×100) is preferably 15% to 75% by volume, more preferably 20% to 60% by volume, and still more preferably 25% to 50% by volume.

<n-Type Organic Semiconductor>

The photoelectric conversion film preferably contains the n-type organic semiconductor in addition to the specific compound 1.

The n-type organic semiconductor is a compound different from the specific compound 1.

The n-type organic semiconductor is an acceptor-property organic semiconductor material (a compound), and refers to an organic compound having a property of easily accepting an electron. That is, the n-type organic semiconductor refers to an organic compound having a large electron affinity of two organic compounds used in contact with each other. That is, any organic compound having an electron accepting property can be used as the acceptor type organic semiconductor.

Examples of the n-type organic semiconductor include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having at least one selected from the group consisting of a nitrogen atom, an oxygen atom, and a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivative and oxadiazole derivatives; anthraquinodimethane derivatives; diphenylquinone derivatives; bathocuproine, bathophenanthroline, and derivatives thereof; triazole compounds; a distyrylarylene derivative; a metal complex having a nitrogen-containing heterocyclic compound as a ligand; a silole compound; and compounds disclosed in paragraphs [0056] to [0057] of JP2006-100767A.

The n-type organic semiconductor (compound) is preferably fullerenes selected from the group consisting of a fullerene and derivatives thereof.

Examples of the fullerenes include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the fullerene derivatives include compounds in which a substituent is added to the above fullerenes. The substituent is preferably an alkyl group, an aryl group, or a heterocyclic group. As the fullerene derivative, the compounds described in JP2007-123707A are preferable.

The n-type organic semiconductor may be an organic coloring agent.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, and a metal complex coloring agent.

The molecular weight of the n-type organic semiconductor is preferably 200 to 1,200, and more preferably 200 to 900.

The maximal absorption wavelength of the n-type organic semiconductor is preferably in a wavelength of 400 nm or less or in a wavelength range of 500 to 600 nm.

It is preferable that the photoelectric conversion film has a bulk hetero structure formed in a state in which the specific compound 1 and the n-type organic semiconductor are mixed. The bulk hetero structure refers to a layer in which the specific compound 1 and the n-type organic semiconductor are mixed and dispersed in the photoelectric conversion film. The photoelectric conversion film having the bulk hetero structure can be formed by either a wet method or a dry method. The bulk hetero structure is described in detail in, for example, paragraphs [0013] and [0014] of JP2005-303266A.

The difference in electron affinity between the specific compound 1 and the n-type organic semiconductor is preferably 0.1 eV or more.

The n-type organic semiconductor may be used alone, or two or more types thereof may be used in combination.

In a case where the photoelectric conversion film contains the n-type organic semiconductor, a content of the n-type organic semiconductor in the photoelectric conversion film (film thickness of n-type organic semiconductor in terms of single layer/film thickness of photoelectric conversion film×100) is preferably 15% to 75% by volume, more preferably 20% to 60% by volume, and still more preferably 20% to 50% by volume.

In a case where the n-type organic semiconductor material includes fullerenes, a content of the fullerenes to a total content of the n-type organic semiconductor material (film thickness of fullerenes in terms of single layer/total film thickness of n-type organic semiconductor materials in terms of single layer×100) is preferably 50% to 100% by volume, and more preferably 80% to 100% by volume. The fullerenes may be used alone, or two or more types thereof may be used in combination.

From the viewpoint of response speed of the photoelectric conversion element, the content of the specific compound 1 to the total content of the specific compound 1 and the n-type organic semiconductor (film thickness in terms of single layer of specific compound 1/(film thickness in terms of single layer of specific compound 1+film thickness in terms of single layer of n-type organic semiconductor)×100) is preferably 20% to 80% by volume, and more preferably 40% to 80% by volume.

In a case where the photoelectric conversion film contains an n-type organic semiconductor and a p-type organic semiconductor, the content of the specific compound 1 (=film thickness in terms of single layer of specific compound 1/(film thickness in terms of single layer of specific compound 1+film thickness in terms of single layer of n-type organic semiconductor+film thickness in terms of single layer of p-type organic semiconductor)×100) is preferably 15% to 75% by volume, and more preferably 30% to 75% by volume.

It is preferable that the photoelectric conversion film is substantially formed of the specific compound 1, the n-type organic semiconductor, and the p-type organic semiconductor included as desired. The term “substantially” indicates that the total content of the specific compound 1, the n-type organic semiconductor, and the p-type organic semiconductor is 90% to 100% by volume, preferably 95% to 100% by volume, and more preferably 99% to 100% by volume, with respect to the total mass of the photoelectric conversion film.

<p-Type Organic Semiconductor>

The photoelectric conversion film preferably contains the p-type organic semiconductor in addition to the specific compound 1.

The p-type organic semiconductor is a compound different from the specific compound 1.

The p-type organic semiconductor is a donor organic semiconductor material (a compound), and refers to an organic compound having a property of easily donating an electron. That is, the p-type organic semiconductor means an organic compound having a smaller ionization potential in a case where two organic compounds are used in contact with each other.

The p-type organic semiconductor may be used alone, or two or more types thereof may be used in combination.

Examples of the p-type organic semiconductor include triarylamine compounds (for example, N,N′-bis(3-methylphenyl)-(1,1′-biphenyl)-4,4′-diamine (TPD), 4,4′-bis[N-(naphthyl)-N-Phenyl-amino]biphenyl (α-NPD), compounds disclosed in paragraphs [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in paragraphs [0044] to [0051] of JP2015-153910A, and compounds disclosed in paragraphs [0086] to [0090] of JP2012-094660A), pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1]benzothieno[3,2-b]thiophene (BTBT) derivative, a thieno[3,2-f:4,5-f]bis[1]benzothiophene (TBBT) derivative, compounds disclosed in paragraphs [0031] to [0036] of JP2018-014474A, compounds disclosed in paragraphs [0043] to [0045] of WO2016-194630A, compounds disclosed in paragraphs [0025] to [0037], and [0099] to [0109] of WO2017-159684A, compounds disclosed in paragraphs [0029] to [0034] of JP2017-076766A, compounds disclosed in paragraphs [0015] to [0025] of WO2018-207722A, compounds disclosed in paragraphs [0045] to [0053] of JP2019-054228A, compounds disclosed in paragraphs [0045] to [0055] of WO2019-058995A, compounds disclosed in paragraphs [0063] to [0089] of WO2019-081416A, compounds disclosed in paragraphs [0033] to [0036] of JP2019-80052A, compounds disclosed in paragraphs [0044] to [0054] of WO2019-054125A, compounds disclosed in paragraphs [0041] to [0046] of WO2019-093188A), compounds in paragraphs [0034] to [0037] of JP2019-050398A, compounds in paragraphs [0033] to [0036] of JP2018-206878A, compounds in paragraph [0038] of JP2018-190755A, compounds in paragraphs [0019] to [0021] of JP2018-026559A, compounds in paragraphs [0031] to [0056] of JP2018-170487A, compounds in paragraphs [0036] to [0041] of JP2018-078270A, compounds in paragraphs [0055] to [0082] of JP2018-166200A, compounds in paragraphs [0041] to [0050] of JP2018-113425A, compounds in paragraphs [0044] to [0048] of JP2018-085430A, compounds in paragraphs [0041] to [0045] of JP2018-056546A, compounds in paragraphs [0042] to [0049] of JP2018-046267A, compounds in paragraphs [0031] to [0036] of JP2018-014474A, compounds disclosed in paragraphs [0036] to [0046] of WO2018-016465A, compounds disclosed in paragraphs [0045] to [0048] of JP2020-010024A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, a fluoranthene derivative, and the like), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands.

Examples of the p-type organic semiconductor also include compounds having an ionization potential smaller than that of the n-type organic semiconductor, and in a case where this condition is satisfied, the organic coloring agents exemplified as the n-type organic semiconductor can be used.

The compounds that can be used as the p-type organic semiconductor compound are exemplified below.

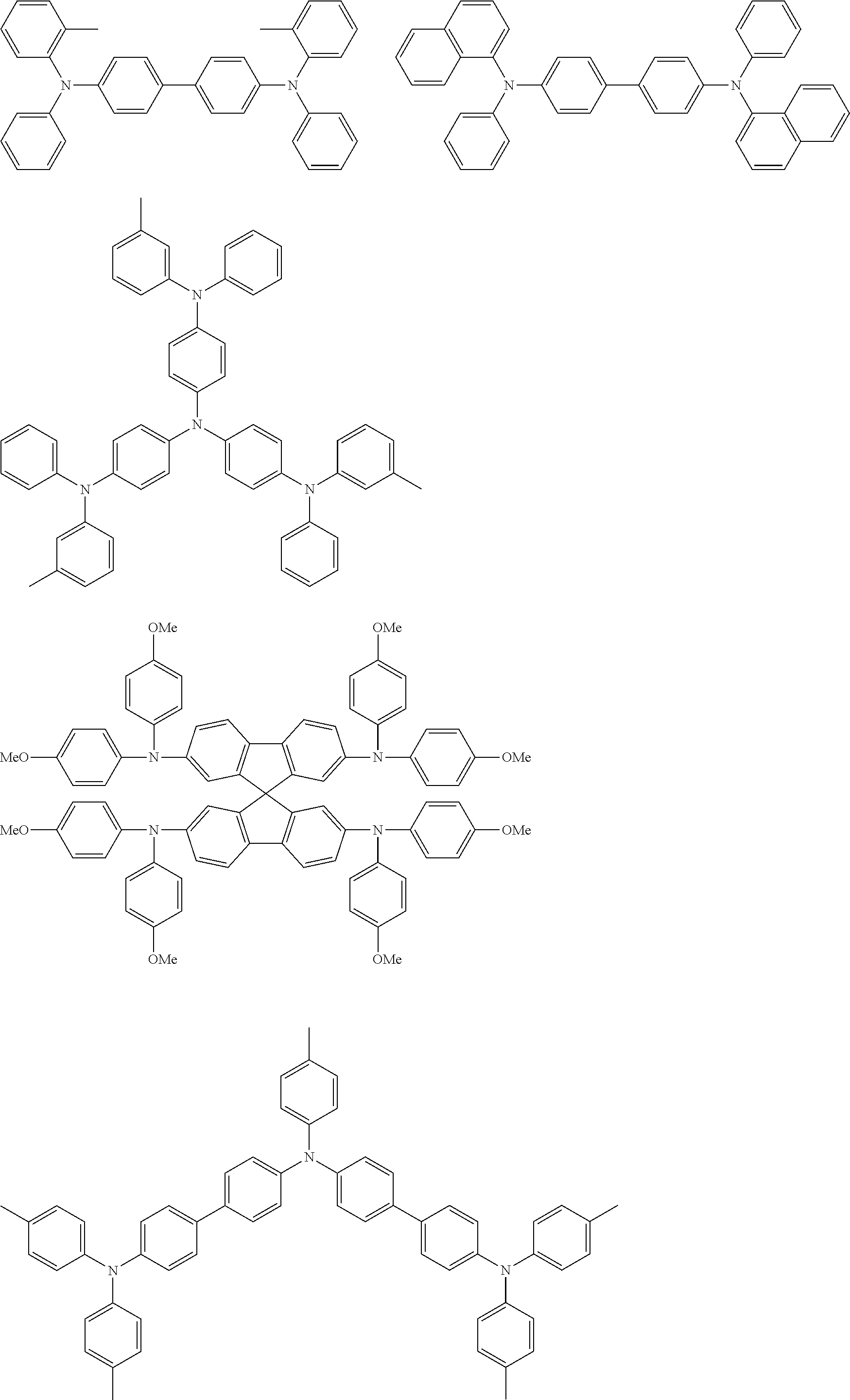

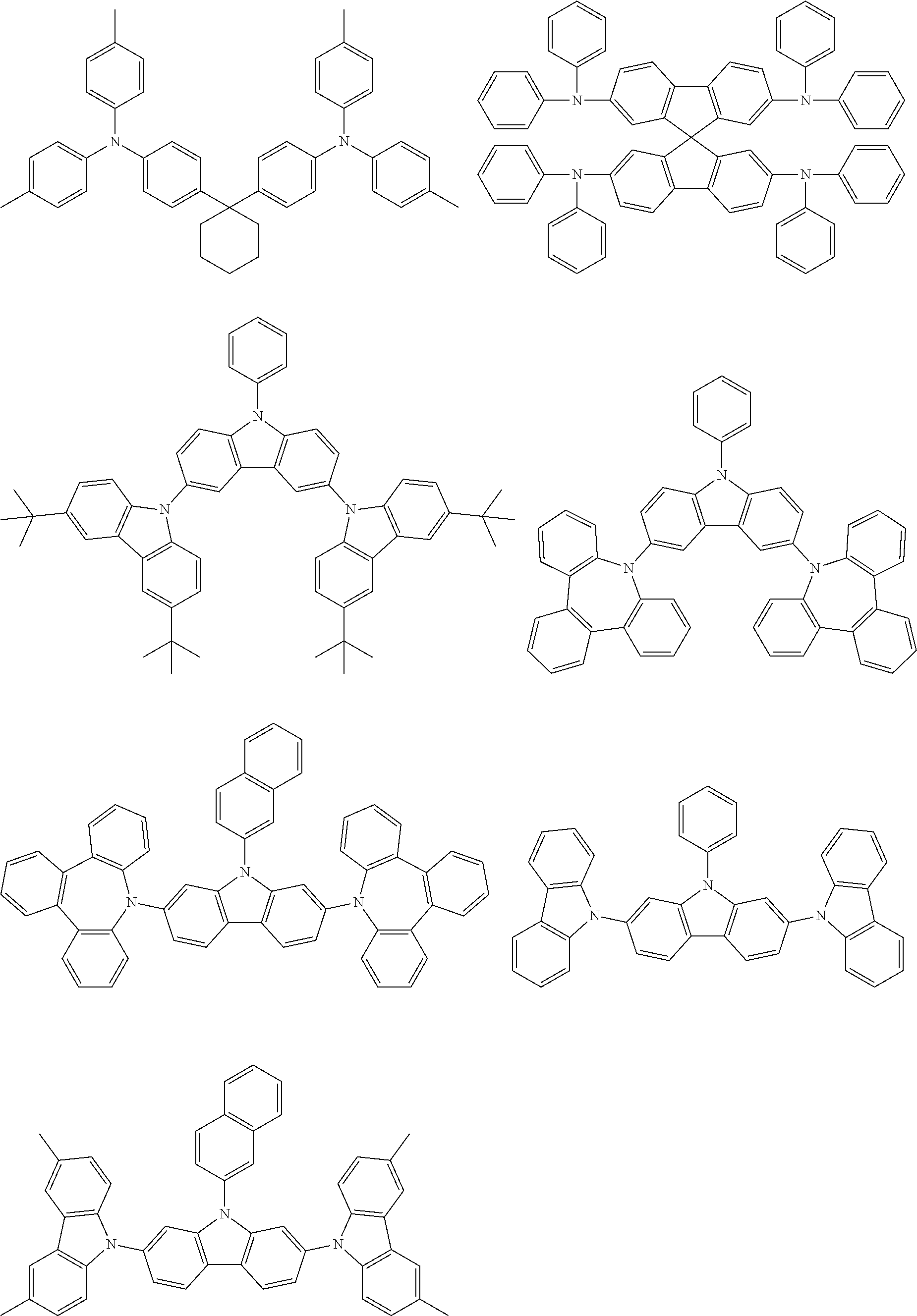

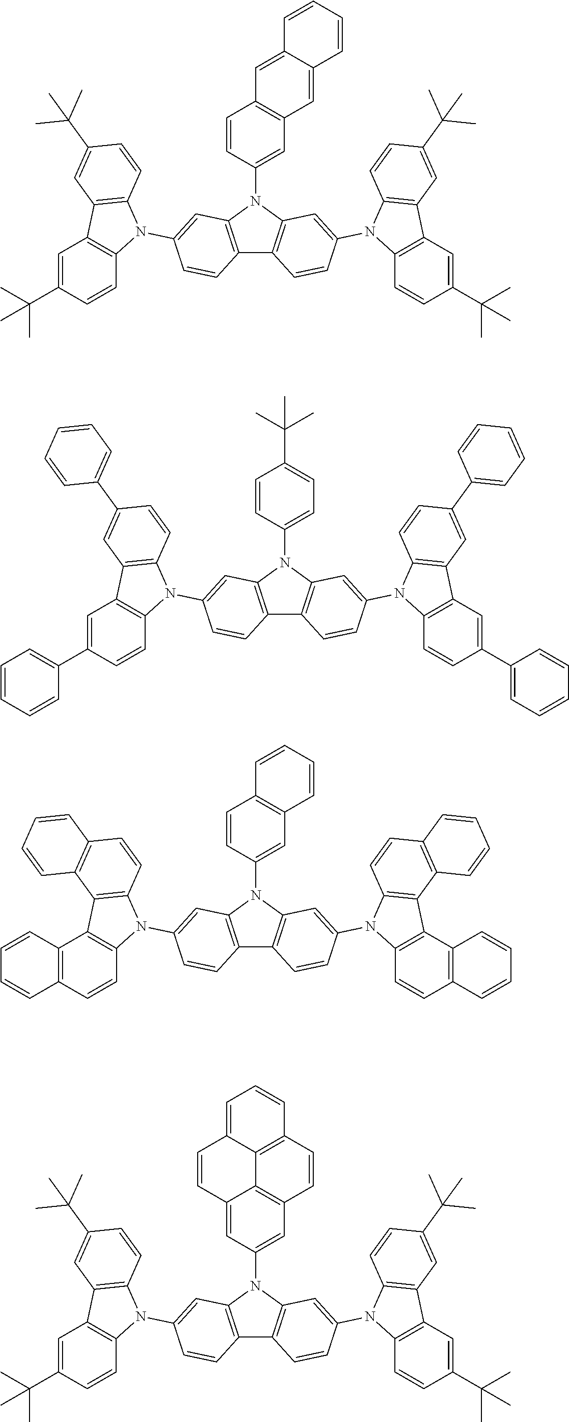

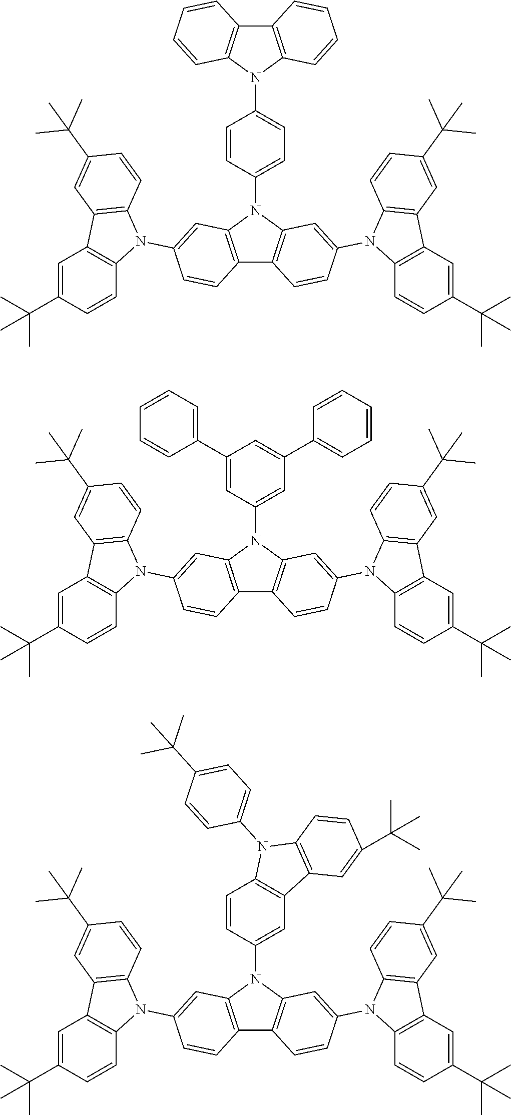

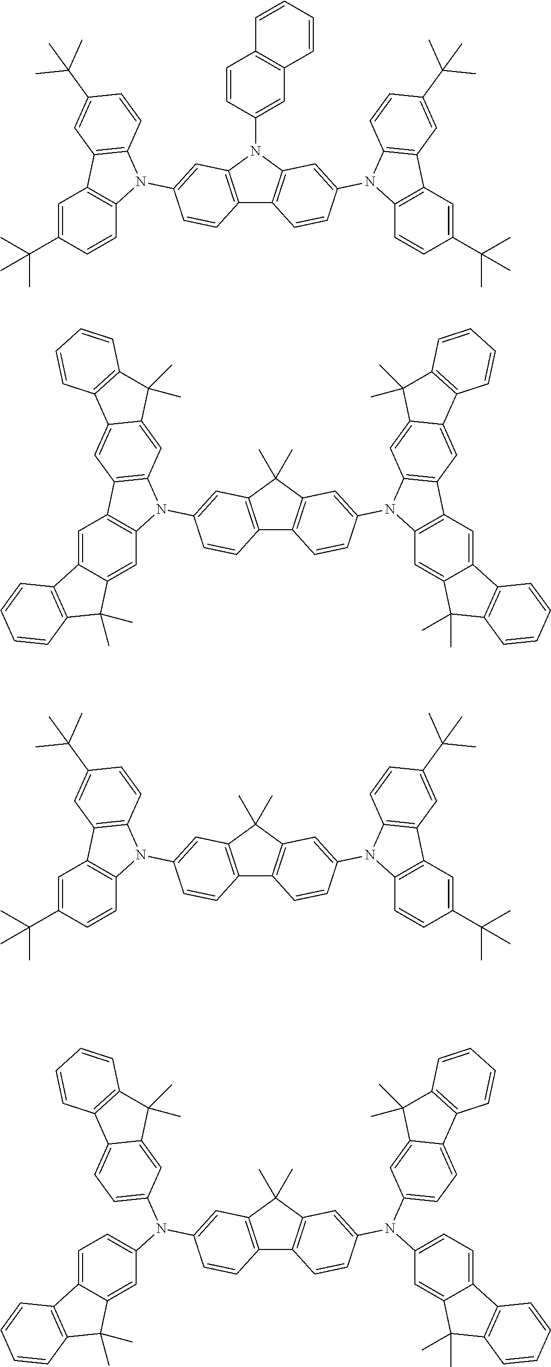

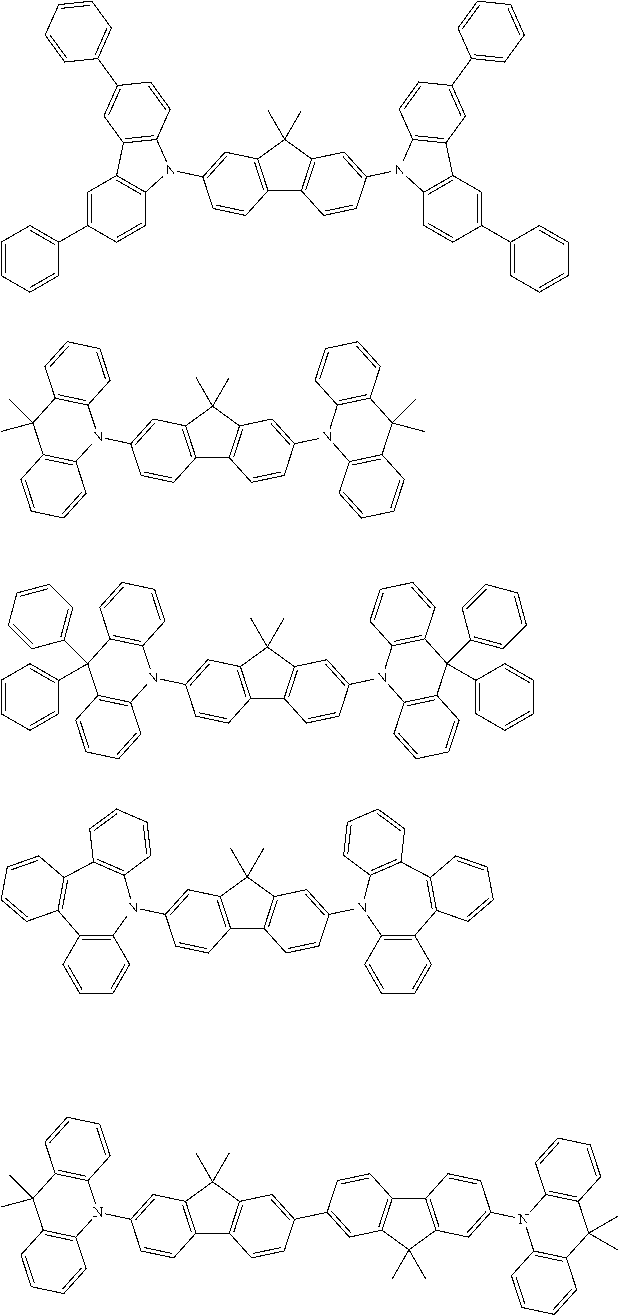

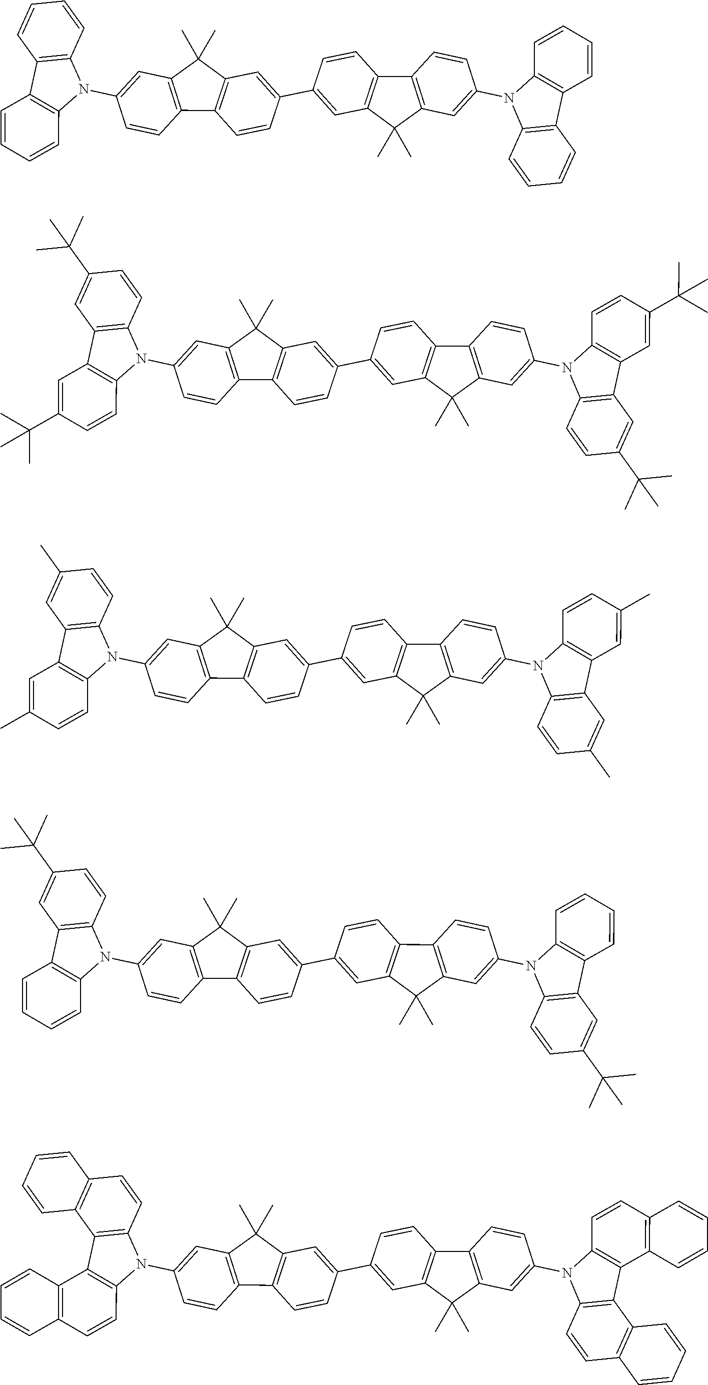

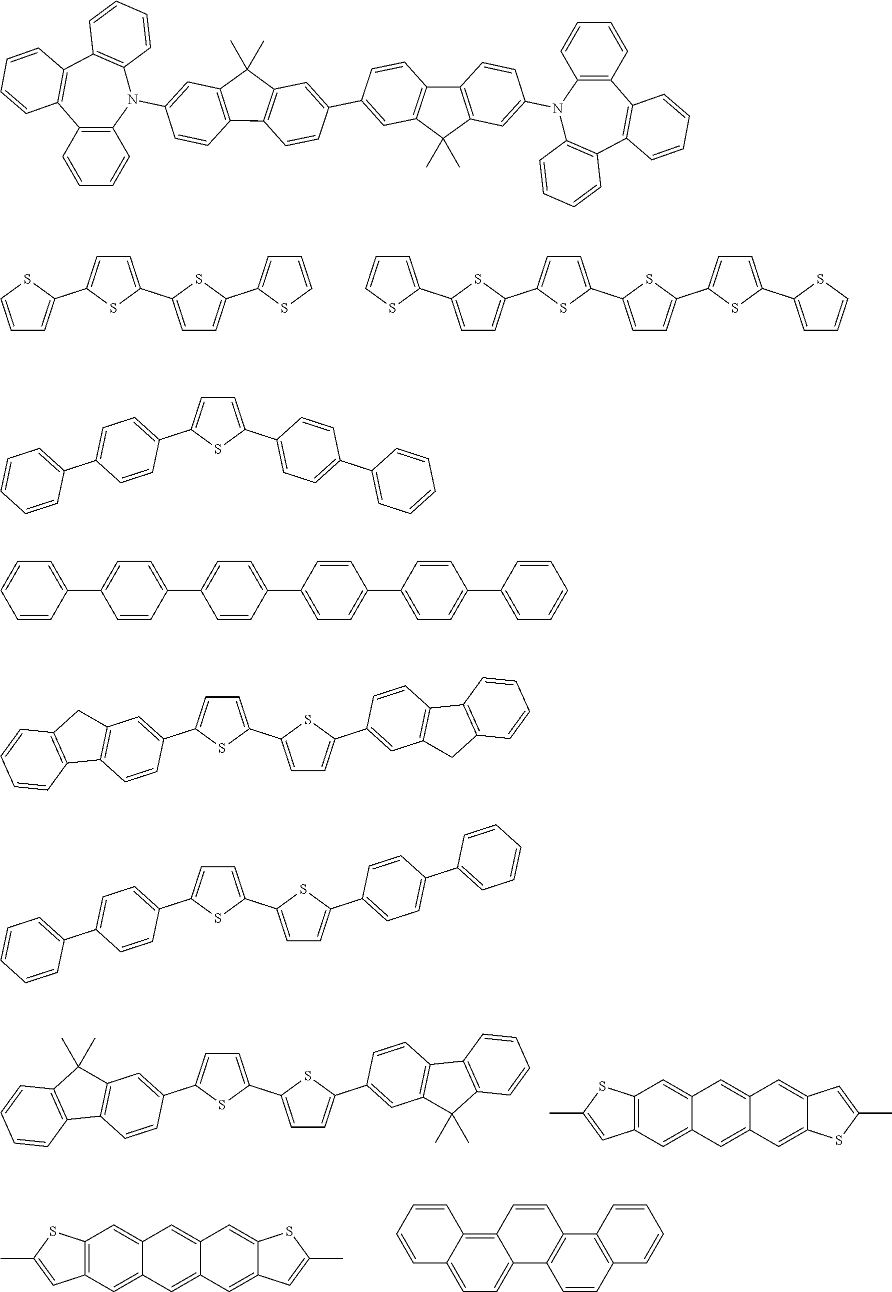

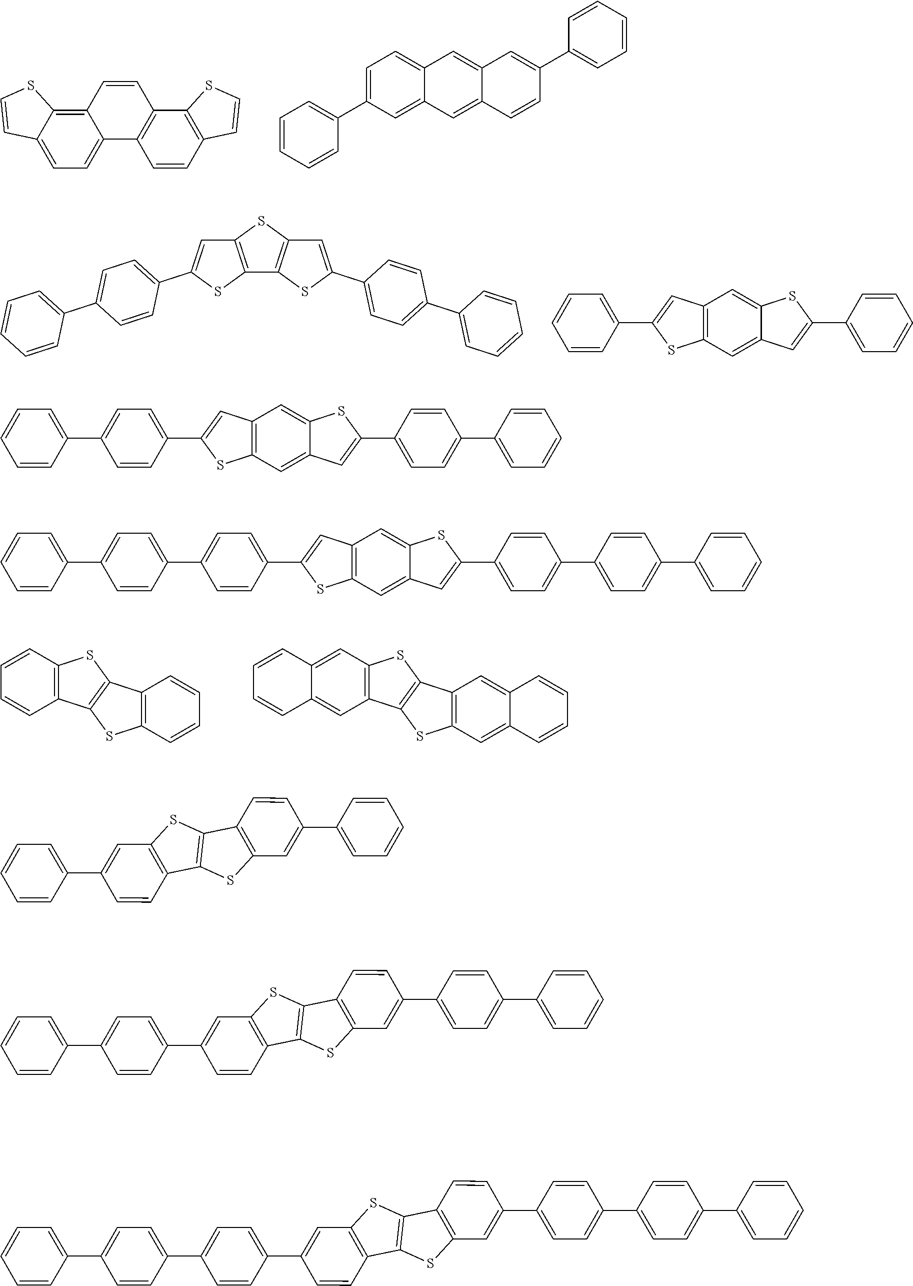

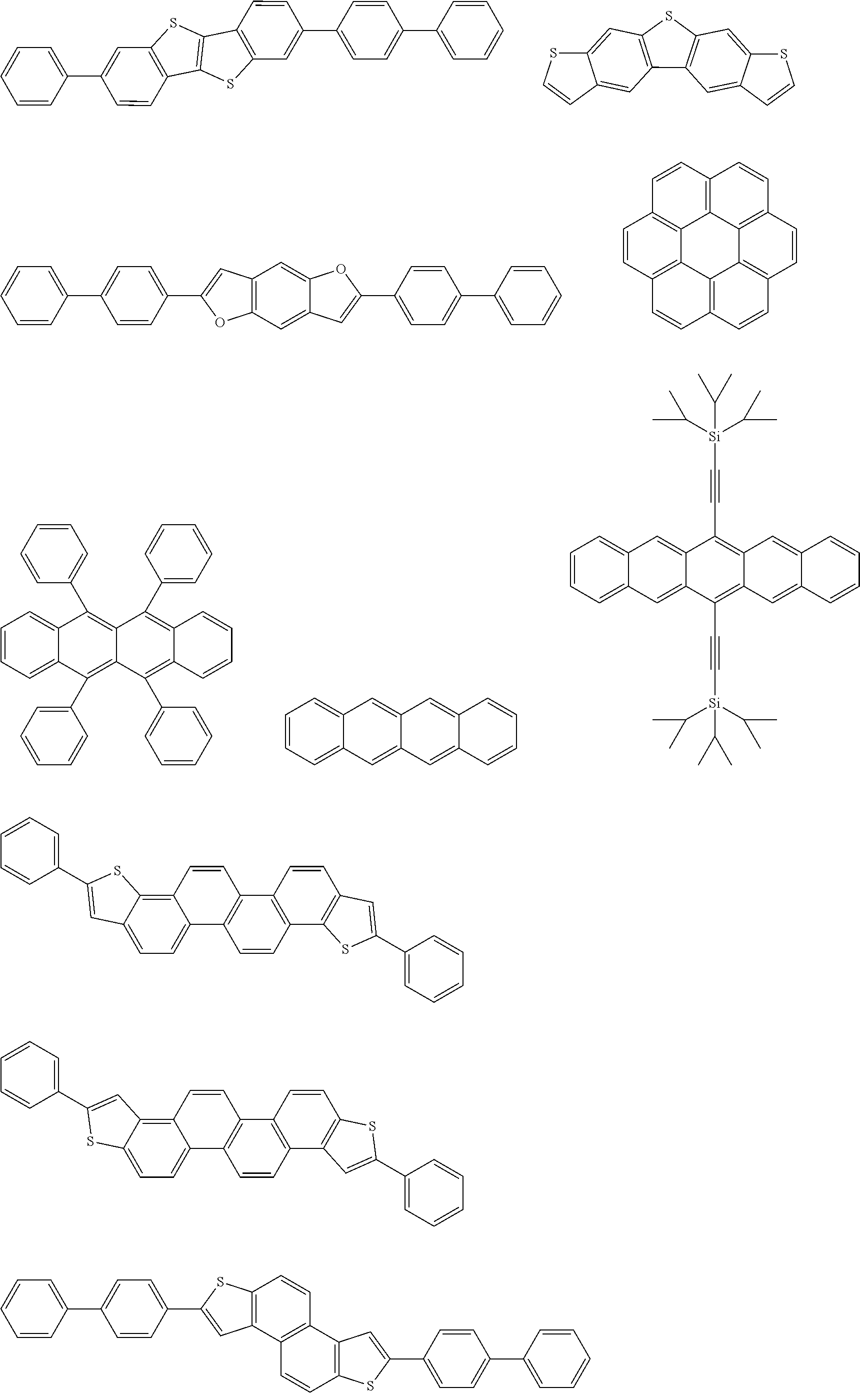

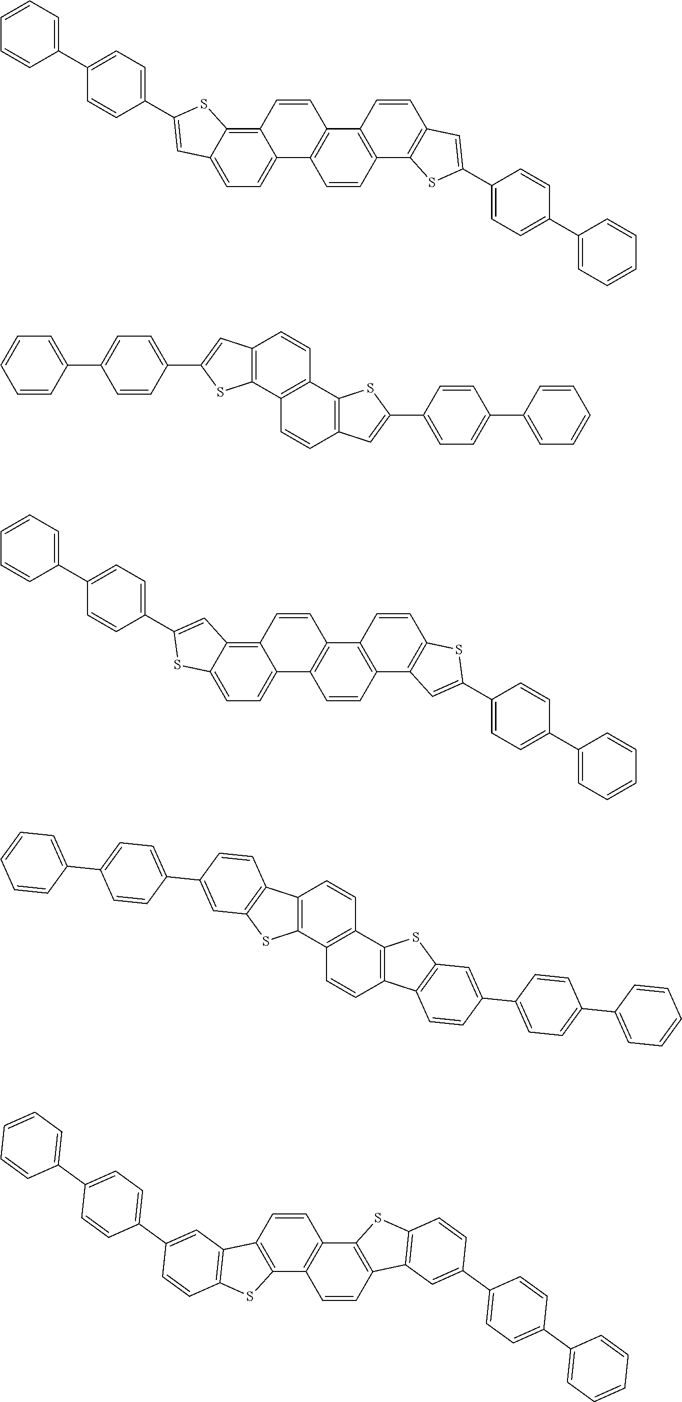 [SMILES: c1ccc(-c2ccc(-c3cc4ccc5c6ccc7c(ccc8cc(-c9ccc(-c%10ccccc%10)cc9)sc87)c6ccc5c4s3)cc2)cc1 ; c1ccc(-c2ccc(-c3cc4ccc5c(ccc6cc(-c7ccc(-c8ccccc8)cc7)sc65)c4s3)cc2)cc1 ; c1ccc(-c2ccc(-c3cc4c(ccc5c4ccc4c6ccc7sc(-c8ccc(-c9ccccc9)cc8)cc7c6ccc54)s3)cc2)cc1 ; c1ccc(-c2ccc(-c3ccc4c(c3)sc3c4ccc4c3ccc3c5ccc(-c6ccc(-c7ccccc7)cc6)cc5sc34)cc2)cc1 ; c1ccc(-c2ccc(-c3ccc4sc5c(ccc6c5ccc5c7cc(-c8ccc(-c9ccccc9)cc8)ccc7sc56)c4c3)cc2)cc1]

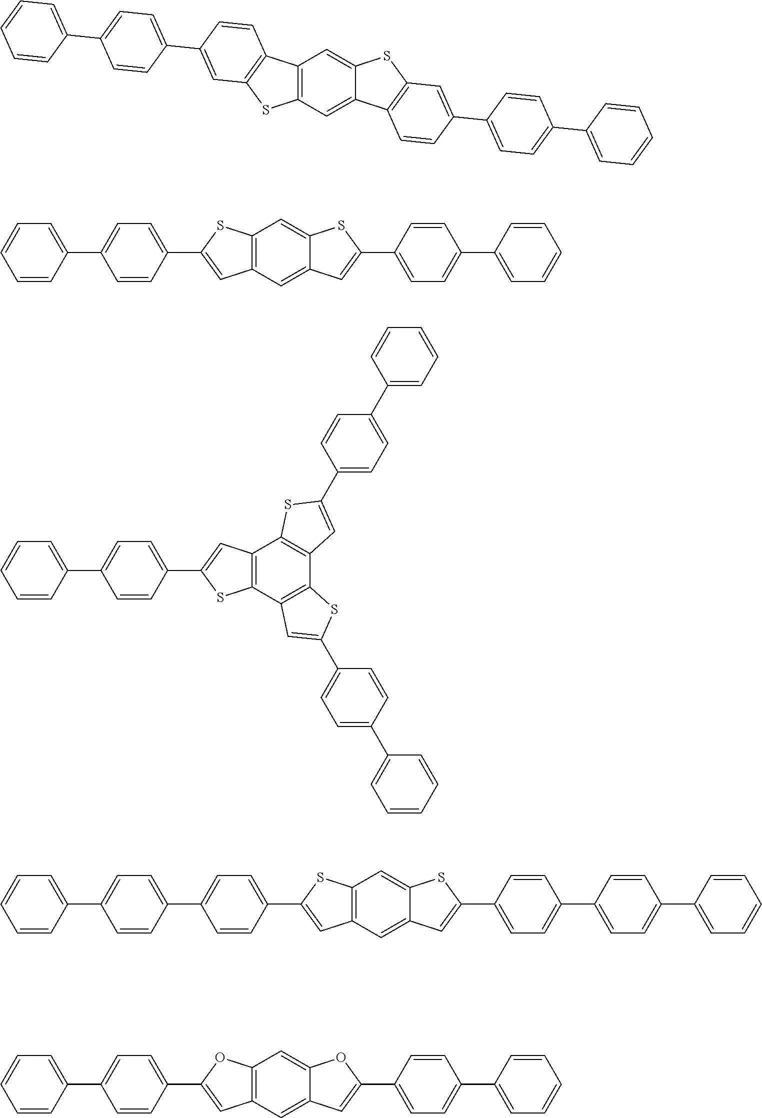 [SMILES: c1ccc(-c2ccc(-c3ccc4c(c3)sc3cc5c(cc34)sc3cc(-c4ccc(-c6ccccc6)cc4)ccc35)cc2)cc1 ; c1ccc(-c2ccc(-c3cc4cc5cc(-c6ccc(-c7ccccc7)cc6)sc5cc4s3)cc2)cc1 ; c1ccc(-c2ccc(-c3cc4c(s3)c3cc(-c5ccc(-c6ccccc6)cc5)sc3c3cc(-c5ccc(-c6ccccc6)cc5)sc43)cc2)cc1 ; c1ccc(-c2ccc(-c3ccc(-c4cc5cc6cc(-c7ccc(-c8ccc(-c9ccccc9)cc8)cc7)sc6cc5s4)cc3)cc2)cc1 ; c1ccc(-c2ccc(-c3cc4cc5cc(-c6ccc(-c7ccccc7)cc6)oc5cc4o3)cc2)cc1]

The difference in the ionization potential between the specific compound 1 and the p-type organic semiconductor is preferably 0.1 eV or more.

The p-type semiconductor material may be used alone, or two or more types thereof may be used in combination.

In a case where the photoelectric conversion film contains the p-type organic semiconductor, a content of the p-type organic semiconductor in the photoelectric conversion film (film thickness of p-type organic semiconductor in terms of single layer/film thickness of photoelectric conversion film×100) is preferably 15% to 75% by volume, more preferably 20% to 60% by volume, and still more preferably 25% to 50% by volume.

The photoelectric conversion film containing the specific compound 1 is a non-light emitting film, and has a feature different from organic light emitting diodes (OLEDs). The non-light emitting film means a film having a light emission quantum efficiency of 1% or less, and the light emission quantum efficiency is preferably 0.5% or less, and more preferably 0.1% or less. The lower limit thereof is often 0% or more.

### <Film Formation Method>

Examples of a film formation method of the photoelectric conversion film include a dry film formation method.

Examples of the dry film formation method include a physical vapor deposition method such as a vapor deposition method (particularly, a vacuum vapor deposition method), a sputtering method, an ion plating method, and a molecular beam epitaxy (MBE) method, and a chemical vapor deposition (CVD) method such as plasma polymerization, and the vacuum vapor deposition method is preferable. In a case where the photoelectric conversion film is formed by the vacuum vapor deposition method, manufacturing conditions such as a degree of vacuum and a vapor deposition temperature can be set according to the normal method.

The film thickness of the photoelectric conversion film is preferably 10 to 1000 nm, more preferably 50 to 800 nm, and still more preferably 50 to 500 nm.

### [Electrode]

The photoelectric conversion element preferably has an electrode.

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the materials constituting the upper electrode 15 include conductive metal oxides such as tin oxide (antimony tin oxide (ATO) and fluorine doped tin oxide (FTO)) doped with antimony, fluorine or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole; and nano carbon materials such as carbon nanotubes, graphene, and the like. From the viewpoint of high conductivity and transparency, conductive metal oxides are preferable.

Generally, in a case where the conductive film is made thinner than a certain range, the resistance value rapidly increases in many cases. In the solid-state imaging element in which the photoelectric conversion element according to the present embodiment is incorporated, the sheet resistance may be 100 to 10,000Ω/□, and the degree of freedom of the film thickness range that can be reduced is large.

In addition, as the film thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the thickness of the upper electrode 15 is preferably 5 to 100 nm, and more preferably 5 to 20 nm.

There is a case where the lower electrode 11 has transparency or an opposite case where the lower electrode 11 does not have transparency and reflects light, depending on the application. Examples of a material constituting the lower electrode 11 include conductive metal oxides such as tin oxide (ATO and FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum; conductive compounds (for example, titanium nitride (TiN)) such as oxides or nitrides of these metals; mixtures or laminates of these metals and conductive metal oxides; organic conductive materials such as polyaniline, polythiophene, and polypyrrole; and carbon materials such as carbon nanotubes and graphene.

The method of forming electrodes can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (such as a sol-gel method), and a coating method with a dispersion of indium tin oxide.

### [Charge Blocking Film: Electron Blocking Film and Positive Hole Blocking Film]

The photoelectric conversion element preferably includes one or more interlayers between the conductive film and the transparent conductive film, in addition to the photoelectric conversion film.

An example of the interlayer includes a charge blocking film. In a case where the photoelectric conversion element has this film, the characteristics (such as photoelectric conversion efficiency and response speed) of the photoelectric conversion element to be obtained are more excellent. Examples of the charge blocking film include an electron blocking film and a positive hole blocking film.

### <Electron Blocking Film>

The electron blocking film is a donor organic semiconductor material (a compound), and the p-type organic semiconductor described above can be used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the photoelectric conversion efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### <Positive Hole Blocking Film>

A positive hole blocking film is an acceptor-property organic semiconductor material (a compound), and the n-type organic semiconductor described above can be used.

In addition, the positive hole blocking film may be formed with a plurality of films.

Examples of a method of producing a charge blocking film include a dry film formation method and a wet film formation method. Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning.

Each film thickness of the charge blocking films (the electron blocking film and the positive hole blocking film) is preferably 3 to 200 nm, more preferably 5 to 100 nm, and still more preferably 5 to 30 nm.

### <Substrate>

The photoelectric conversion element may further include a substrate.

Examples of the substrate include a semiconductor substrate, a glass substrate, and a plastic substrate.

As a position of the substrate, in general, the conductive film, the photoelectric conversion film, and the transparent conductive film are laminated on the substrate in this order.

### <Sealing Layer>

The photoelectric conversion element may further include a sealing layer.

The performance of the photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, metal nitride, or metal nitride oxide which are dense and into which water molecules do not permeate.

Examples of the sealing layer include compounds described in paragraphs to of JP2011-082508A, the contents of which are incorporated herein by reference.

Hereinafter, the form of each layer constituting the photoelectric conversion element according to the second embodiment of the present invention will be described in detail.

### <<Photoelectric Conversion Element According to Second Embodiment>>

The photoelectric conversion element according to the second embodiment is a photoelectric conversion element including, in the following order, a conductive film, a photoelectric conversion film, and a transparent conductive film, in which the photoelectric conversion film contains the specific compound 2.

The photoelectric conversion element according to the second embodiment is the same as the photoelectric conversion element according to the first embodiment, except that the photoelectric conversion film contains the specific compound 2 instead of the specific compound 1, and the suitable ranges thereof are also the same.

Specifically, the photoelectric conversion element according to the second embodiment may include the electrode and the charge blocking film (for example, an electron blocking film, a positive hole blocking film, and the like), which can be included in the photoelectric conversion element according to the first embodiment. In addition, the description of the “specific compound 1” in the photoelectric conversion element according to the first embodiment may be read as “specific compound 2”. For example, the description of “the molecular weight of the specific compound 1 is preferably 400 to 1,200” may be read as “the molecular weight of the specific compound 2 is preferably 400 to 1,200”.

### [Photoelectric Conversion Film]

The photoelectric conversion element according to the second embodiment has a photoelectric conversion film.

<Specific compound 2>

The photoelectric conversion film contains a specific compound 2.

D²=A² (2)

In Formula (2), A² represents a group represented by any of Formulae (A-1) to (A-4). D² represents a group represented by Formula (D-2).

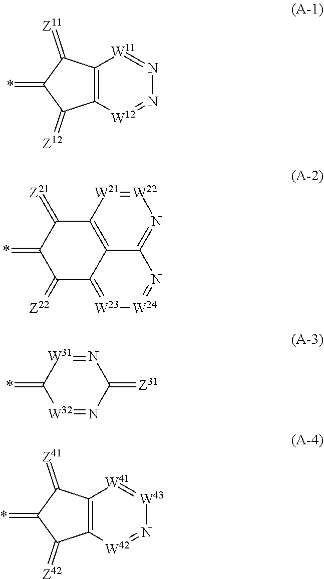

Each notation in Formulae (A-1) to (A-3) is the same as each notation in the specific compound 1, and the suitable range thereof is also the same.

In Formula (A-4), * represents a bonding position. W⁴¹ to W⁴³ each independently represent —CR^{W41}═ or a nitrogen atom. R^{W41} represents a hydrogen atom or a substituent. Z⁴¹ and Z⁴² each independently represent an oxygen atom, a sulfur atom, a selenium atom, =NR^{Z41}, or —C(R^{Z42})(R^{Z43}). R^{Z41} to R^{Z43} each independently represent a hydrogen atom or a substituent.

In Formula (A-4), W⁴¹ to W⁴³ each independently represent —CR^{W41}═ or a nitrogen atom. R^{W41} represents a hydrogen atom or a substituent.

It is preferable that at least one of R^{W41}, R^{W42}, or R^{W43} represents —CR^{W41}═, and it is more preferable that W⁴¹, W⁴², or W⁴³ represent —CR^{W41}═.

Examples of the above-described substituent include a group exemplified by the substituent W.

R^{W41} is preferably a hydrogen atom.

In a case where a plurality of R^{W41}'s are present, R^{W41}'s may be the same or different from each other.

In Formula (2), D² represents a group represented by Formula (D-2).

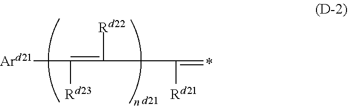

In Formula (D-2), * represents a bonding position. Ar^{d21} represents a group represented by Formula (Ar-1). R^{d21} to R^{d23} each independently represent a hydrogen atom or a substituent. n^{d21} represents an integer of 0 to 5.

The group represented by Formula (Ar-1) has the same meaning as the group represented by Formula (Ar-1) as Ar^{d11}, and a suitable aspect thereof is also the same.

In Formula (D-2), R^{d21} to R^{d23} and n^{d21} have the same meanings as R^{d11} to R^{d13} and n^{d11} in Formula (D-1), and suitable aspects thereof are also the same.

In Formulae (A-1) to (A-4), it is preferable that Z¹¹, Z¹², Z²¹, Z²², Z³¹, Z⁴¹, and Z⁴² are an oxygen atom or a sulfur atom.

Examples of the specific compound 2 include the following compounds.

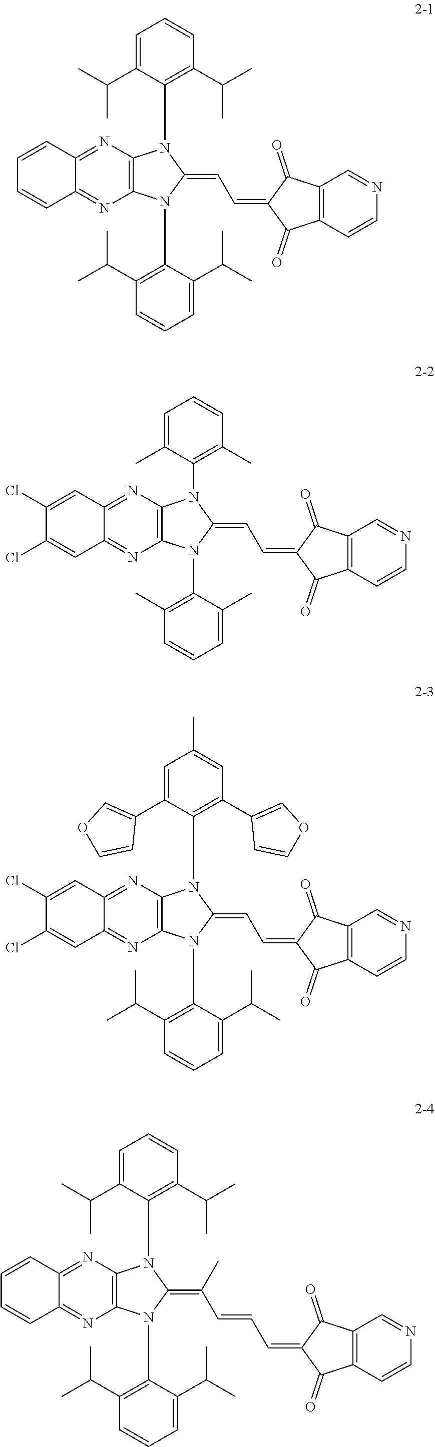 [SMILES: CC(C)c1cccc(C(C)C)c1N1C(=C/C=C2/C(=O)c3ccncc3C2=O)N(c2c(C(C)C)cccc2C(C)C)c2nc3ccccc3nc21 ; Cc1cccc(C)c1N1C(=C/C=C2/C(=O)c3ccncc3C2=O)N(c2c(C)cccc2C)c2nc3cc(Cl)c(Cl)cc3nc21 ; Cc1cc(-c2ccoc2)c(N2/C(=C/C=C3/C(=O)c4ccncc4C3=O)N(c3c(C(C)C)cccc3C(C)C)c3nc4cc(Cl)c(Cl)cc4nc32)c(-c2ccoc2)c1 ; CC(/C=C/C=C1/C(=O)c2ccncc2C1=O)=C1N(c2c(C(C)C)cccc2C(C)C)c2nc3ccccc3nc2N1c1c(C(C)C)cccc1C(C)C]

### [Imaging Element]

An example of the application of the photoelectric conversion element includes an imaging element.

The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

### [Optical Sensor]

Examples of another application of the photoelectric conversion element include the photoelectric cell and the optical sensor, but the photoelectric conversion element of the embodiment of the present invention is preferably used as the optical sensor. The photoelectric conversion element may be used alone as the optical sensor. Alternately, the photoelectric conversion element may be used as a line sensor in which the photoelectric conversion elements are linearly arranged or as a two-dimensional sensor in which the photoelectric conversion elements are arranged on a plane.

### [Compound]

The present invention further includes the invention of compounds. The compounds according to the embodiment of the present invention are the specific compound 1 and the specific compound 2.

### Examples

Hereinafter, the present invention will be described in more detail based on Examples. The material, the amount used, the proportion, the process contents, the process procedure, and the like shown in the following examples can be appropriately changed, within a range not departing from the gist of the present invention. Therefore, the scope of the present invention should not be construed as being limited to Examples shown below.

### [Compound Used for Photoelectric Conversion Film]

### [Synthesis of Compound D-1]

Compound D-1 was synthesized according to the following scheme.

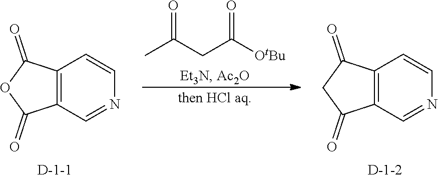

3,4-pyridinedicarboxylic anhydride (3 g, 20 mmol), acetic acid anhydride (30 mL), triethylamine (5.6 mL, 40 mmol), and acetoacetic acid tert-butyl (3.3 mL, 20 mmol) were mixed with each other and stirred at room temperature for 24 hours. The acetic anhydride was distilled away under reduced pressure to obtain an intermediate. Next, water (18 mL) and a 30% by mass aqueous hydrochloric acid solution (12 mL) were added thereto, and the mixture was stirred at room temperature for 1 hour. The reaction solution was cooled with ice, the generated precipitate was filtered, washed with water, and then dried to obtain 5H-cyclopenta[c]pyridine-5,7(6H)-dione (Compound D-1-2) (2.97 g, yield of 100%).

The results of identifying the obtained Compound D-1-2 by nuclear magnetic resonance (NMR) are shown below. 1H-NMR (DMSO-d₆, 400 MHz) δ=3.17 (2H, s), 7.88 (1H, s), 9.11 (1H, d), 9.27 (1H, s).

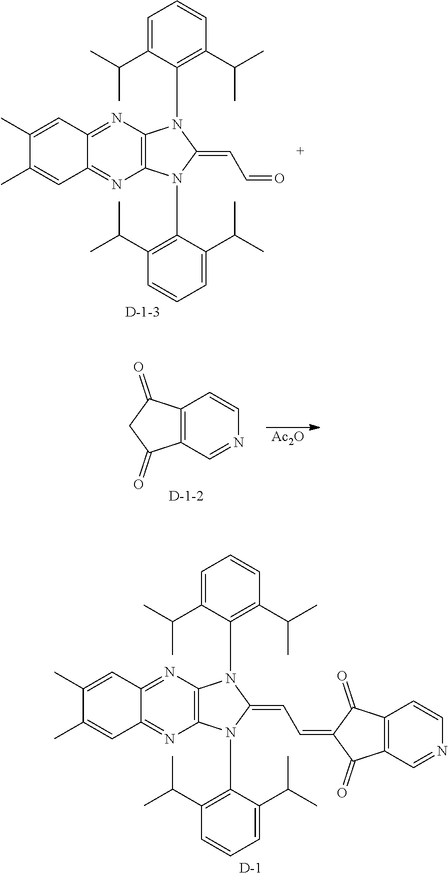

Compound D-1-3 (2 g, 2.9 mmol), Compound D-1-2 (1.03 g, 7.2 mmol), and acetic anhydride (20 mL) were mixed with each other, and the mixture was heated and stirred at 110° C. for 2.5 hours. Furthermore, Compound D-1-2 (213 mg, 1.4 mmol) was added thereto, and the mixture was heated and stirred at 110° C. for 2 hours. The reaction solution was cooled with ice, the generated precipitate was filtered, and washed with methanol. The obtained crude product was purified by silica gel column chromatography (eluent (volume ratio) of dichloromethane:ethyl acetate=9:1 to 7:3), and then crystallized (dichloromethane:acetonitrile) to obtain Compound D-1 (1.27 g, yield of 53%).

The results of identifying the obtained Compound D-1 by NMR are shown below. 1H-NMR (CDCl₃, 400 MHz) δ=0.78 (6H, d), 0.81 (6H, d), 1.53 (2H, brs), 1.93 (12H, s), 2.44 (3H, s), 2.51 (3H, s), 2.66 (3H, s), 6.70 (2H, brs), 6.78 (2H, s), 6.91 (4H, s), 7.32 (2H, d), 7.49 (3H, $), 7.60 (1H, s), 7.66 (1H, s), 7.95 (1H, s), 8.80 (1H, dd), 8.90 (1H, d).

A compound used in the photoelectric conversion film, other than Compound D-1 was synthesized with reference to the synthesis method of Compound D-1.

Compound D-1 and Compound D-4 correspond to the specific compound 2, and all of Compounds D-2 to D-3 and Compounds D-5 to D-11 correspond to the specific compound 1. Both Compound R-1 and Compound R-2 did not correspond to the specific compound.

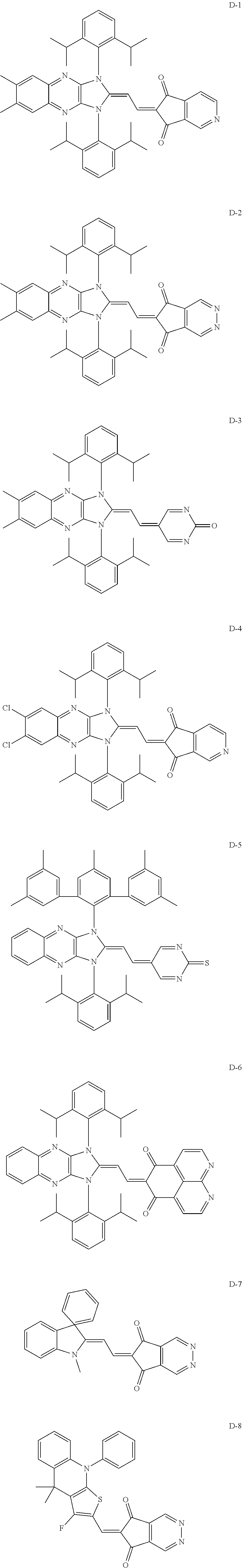

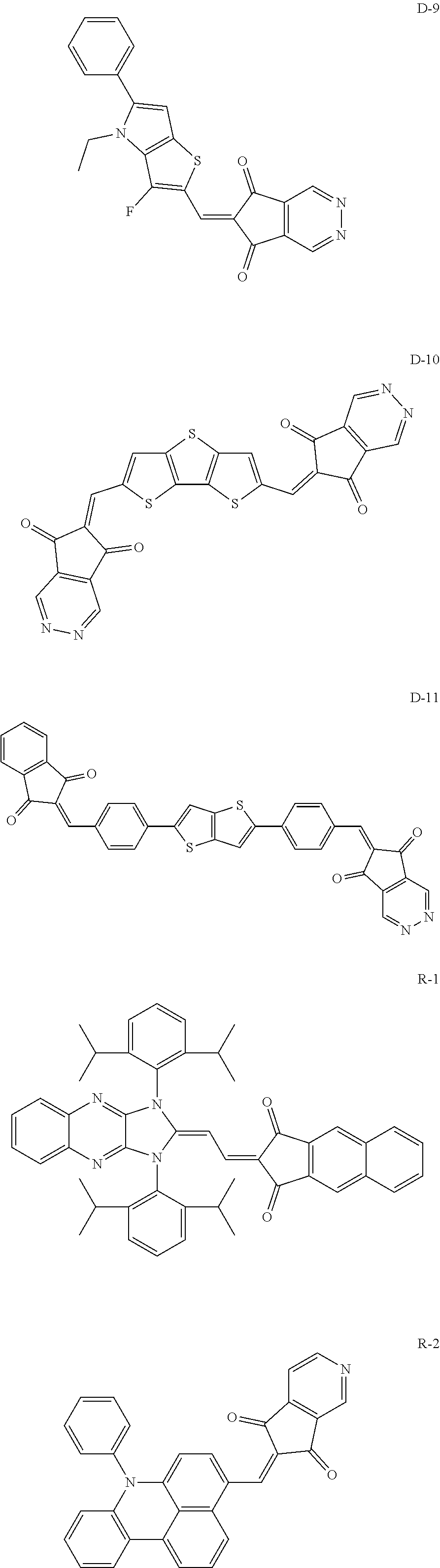 [SMILES: CCn1c(-c2ccccc2)cc2sc(C=C3C(=O)c4cnncc4C3=O)c(F)c21 ; O=C1C(=Cc2cc3sc4cc(C=C5C(=O)c6cnncc6C5=O)sc4c3s2)C(=O)c2cnncc21 ; O=C1C(=Cc2ccc(-c3cc4sc(-c5ccc(C=C6C(=O)c7cnncc7C6=O)cc5)cc4s3)cc2)C(=O)c2ccccc21 ; CC(C)c1cccc(C(C)C)c1N1C(=CC=C2C(=O)c3cc4ccccc4cc3C2=O)N(c2c(C(C)C)cccc2C(C)C)c2nc3ccccc3nc21 ; O=C1/C(=C\c2ccc3c4c(cccc24)-c2ccccc2N3c2ccccc2)C(=O)c2cnccc21]

[n-Type Organic Semiconductor]
C₆₀: fullerene (C₆₀)
   [p-Type Organic Semiconductor]

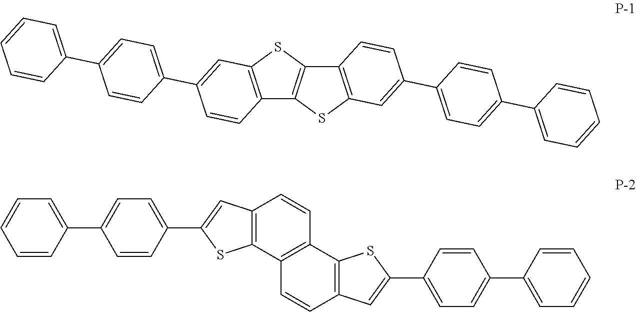 [SMILES: c1ccc(-c2ccc(-c3ccc4c(c3)sc3c5ccc(-c6ccc(-c7ccccc7)cc6)cc5sc43)cc2)cc1 ; c1ccc(-c2ccc(-c3cc4ccc5c(ccc6cc(-c7ccc(-c8ccccc8)cc7)sc65)c4s3)cc2)cc1]

### [Evaluation]

### [Production of Photoelectric Conversion Element]

The photoelectric conversion element (A) of the form illustrated in FIG. 2 was produced using the obtained compounds. Here, the photoelectric conversion element includes a lower electrode 11, an electron blocking film 16A, a photoelectric conversion film 12, a positive hole blocking film 16B, and an upper electrode 15.

Specifically, an amorphous ITO was formed into a film on a glass substrate by a sputtering method to form the lower electrode 11 (thickness: 30 nm). Furthermore, Compound C-1 described below was formed into a film on the lower electrode 11 by a vacuum thermal vapor deposition method to form the electron blocking film 16A (thickness: 30 nm). Furthermore, in a state where the temperature of the substrate was controlled to 25° C., each specific compound and the n-type organic semiconductor (fullerene (C₆₀)) were co-vapor deposited on the electron blocking film 16A by a vacuum vapor deposition method, each to be 80 nm in terms of a single layer, thereby forming a film. As a result, a photoelectric conversion film 12 having a bulk hetero structure of 160 nm (240 nm in a case where the p-type organic semiconductor material was also used) was formed. In this case, a film formation rate of the photoelectric conversion film 12 was set to 1.0 Å/sec.

Furthermore, Compound C-2 described below was vapor-deposited on the photoelectric conversion film 12 to form the positive hole blocking film 16B (thickness: 10 nm). Amorphous ITO was formed into a film on the positive hole blocking film 16B by a sputtering method to form the upper electrode 15 (the transparent conductive film) (thickness: 10 nm). After the SiO film was formed as the sealing layer on the upper electrode 15 by a vacuum vapor deposition method, an aluminum oxide (Al₂O₃) layer was formed thereon by an atomic layer chemical vapor deposition (ALCVD) method to produce each photoelectric conversion element and the photoelectric conversion element (A) was produced.

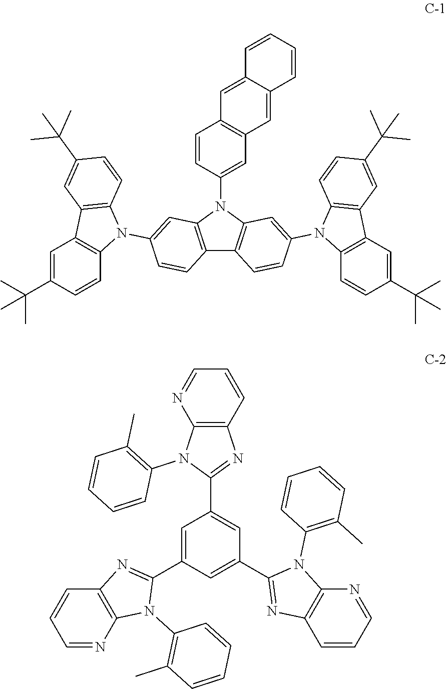 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1 ; Cc1ccccc1-n1c(-c2cc(-c3nc4cccnc4n3-c3ccccc3C)cc(-c3nc4cccnc4n3-c3ccccc3C)c2)nc2cccnc21]

### [Evaluation of Dark Current]

The dark current of each of the obtained photoelectric conversion elements (A) was measured by the following method.

A voltage was applied to the lower electrode and the upper electrode of each of the photoelectric conversion elements (A) to have an electric field strength of 2.5×10⁵ V/cm and current values (dark current) in a dark place were measured. As a result, it was found that all of the photoelectric conversion elements had a dark current of 50 nA/cm² or less, which indicates that all of the photoelectric conversion elements had a sufficiently low dark current.

### [Evaluation of Photoelectric Conversion Efficiency (External Quantum Efficiency)]

The drive of each photoelectric conversion element (A) thus obtained was confirmed. A voltage was applied to each photoelectric conversion element (A) to have an electric field strength of 2.0×10⁵ V/cm. Thereafter, light was emitted from the upper electrode (transparent conductive film) side, and the measurement of the incident photon-to-current conversion efficiency (IPCE) was performed to calculate the integrated value of the photoelectric conversion efficiency (external quantum efficiency) at a wavelength of 560 nm. The photoelectric conversion efficiency was measured using a constant energy quantum efficiency measuring device manufactured by Optel Co., Ltd. The amount of light emitted was 50 μW/cm². In a case where the integrated value of the photoelectric conversion efficiency of the photoelectric conversion element (A) of Example 1-1 was standardized to 1, the integrated value of the photoelectric conversion efficiency of each photoelectric conversion element (A) was obtained and evaluated according to the following evaluation standard.
AA: 1.1 or more
A: 0.9 or more and less than 1.1
B: 0.8 or more and less than 0.9
C: 0.7 or more and less than 0.8
D: 0.6 or more and less than 0.7
E: less than 0.6

The above-described evaluation result is preferably C or more and most preferably AA.

In addition, it was confirmed that the photoelectric conversion efficiency of all the photoelectric conversion elements (A) of each of Examples and each of Comparative Examples at a wavelength of 560 nm was 40% or more, and the photoelectric conversion elements (A) had an external quantum efficiency by a certain level or more as the photoelectric conversion element.

### [Evaluation of Responsiveness]

The responsiveness of each of the obtained photoelectric conversion elements (A) was evaluated.

A voltage was applied to each photoelectric conversion element to have a strength of 2.0×10⁵ V/cm. Thereafter, light emitting diodes (LEDs) were turned on momentarily to emit light from the upper electrode (transparent conductive film) side, a photocurrent at a wavelength of 560 nm was measured with an oscilloscope, and a rise time from a signal intensity of 0% (when the light is not emitted) to 97% was calculated. Next, the rising time of each photoelectric conversion element (A) in a case where the rising time of the photoelectric conversion element of Example 1-1 was standardized to 1, at a wavelength of 560 nm, was obtained, and the responsiveness of each photoelectric conversion element (A) was evaluated based on the rising time according to the following evaluation standard.
AA: less than 0.9
A: 0.9 or more and less than 2.0
B: 2.0 or more and less than 3.0
C: 3.0 or more and less than 4.0
D: 4.0 or more and less than 5.0
E: 5.0 or more

The above-described evaluation result is preferably C or more and most preferably AA.

### [Evaluation of Manufacturing Suitability]

A photoelectric conversion element (B) of each of Examples and each of Comparative Examples was produced according to the same procedure as that of the photoelectric conversion element (A), except that a film formation rate of the photoelectric conversion film 12 was set to 3.0 Å/sec. The photoelectric conversion efficiency (external quantum efficiency) of the obtained photoelectric conversion element (B) was evaluated by the same method as shown in the section of [Evaluation of photoelectric conversion efficiency (external quantum efficiency)].

The photoelectric conversion efficiencies of the photoelectric conversion element (A) and the photoelectric conversion element (B) in the same configuration of Examples or the same configuration of Comparative Examples were compared to calculate a relative ratio B/A of “the photoelectric conversion efficiency of the photoelectric conversion element (B)/the photoelectric conversion efficiency of the photoelectric conversion element (A)”. The manufacturing suitability of each photoelectric conversion element was evaluated by comparing the obtained values with the following standard. The fact that the present evaluation results is excellent indicates that the compound is a material in which the performance is less likely to be deteriorated during high-speed film formation, and indicates that the compound has excellent manufacturing suitability.
A: 0.9 or more
B: less than 0.9

Hereinafter, the evaluation results are shown in the table.

TABLE 1

| | | | | Photoelectric | | |
|---|---|---|---|---|---|---|
| | | | | conversion | | |
| | | | | efficiency | | |
| | | n-Type organic | p-Type organic | (wavelength | | Manufacturing |
| | Compound | semiconductor | semiconductor | of 560 nm) | Responsiveness | suitability |
| Example 1-1 | D-1 | C₆₀ | — | A | A | A |
| Example 1-2 | D-2 | C₆₀ | — | A | A | A |
| Example 1-3 | D-3 | C₆₀ | — | A | A | A |
| Example 1-4 | D-4 | C₆₀ | — | A | A | A |
| Example 1-5 | D-5 | C₆₀ | — | A | A | A |
| Example 1-6 | D-6 | C₆₀ | — | A | A | A |
| Example 1-7 | D-7 | C₆₀ | — | A | A | A |
| Example 1-8 | D-8 | C₆₀ | — | A | A | A |
| Example 1-9 | D-9 | C₆₀ | — | A | A | A |
| Example 1-10 | D-10 | C₆₀ | — | A | A | A |
| Example 1-11 | D-11 | C₆₀ | — | A | A | A |
| Example 1-12 | D-1 | C₆₀ | P-1 | AA | AA | A |
| Example 1-13 | D-1 | C₆₀ | P-2 | AA | AA | A |
| Comparative | R-1 | C₆₀ | — | A | B | B |
| Example 1-1 | | | | | | |
| Comparative | R-2 | C₆₀ | — | E | E | B |
| Example 1-2 | | | | | | |
| Comparative | R-1 | C₆₀ | P-1 | A | A | B |
| Example 1-3 | | | | | | |

From the results shown in the above table, it was confirmed that the photoelectric conversion element according to the embodiment of the present invention has excellent manufacturing suitability.

In a case where the photoelectric conversion film further contained the n-type organic semiconductor and the p-type organic semiconductor, it was confirmed that both the photoelectric conversion efficiency and the responsiveness were more excellent (Examples 1-1 to 1-13).

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: positive hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1),
$${D}^{1} = {A}^{1} \qquad ( 1 )$$
in Formula (1), A¹ represents a group represented by any of Formulae (A-1) to (A-3) and D¹ represents a divalent organic group, 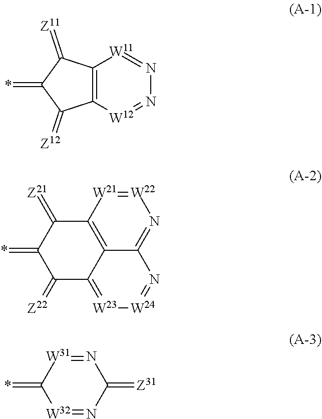
in Formula (A-1), * represents a bonding position, W¹¹ and W¹² each independently represent —CR^{W11}═ or a nitrogen atom, R^{W11} represents a hydrogen atom or a substituent, Z¹¹ and Z¹² each independently represent an oxygen atom, a sulfur atom, a selenium atom, =NR^{Z11}, or ═C(R^{Z12})(R^{Z13}), and R^{Z11} to R^{Z13} each independently represent a hydrogen atom or a substituent,
in Formula (A-2), * represents a bonding position, W²¹ to W²⁴ each independently represent —CR^{W21}═ or a nitrogen atom, R^{W21} represents a hydrogen atom or a substituent, Z²¹ and Z²² each independently represent an oxygen atom, a sulfur atom, a selenium atom, =NR^{Z21}, or =C(R^{Z22})(R^{Z23}), and R^{Z21} to R^{Z23} each independently represent a hydrogen atom or a substituent,
in Formula (A-3), * represents a bonding position, W³¹ and W³² each independently represent —CR^{W31}═ or a nitrogen atom, R^{W31} represents a hydrogen atom, a halogen atom, a cyano group, an aromatic ring group which may have a substituent, an aliphatic hydrocarbon group which may have a substituent, —OR^{W32}, —SR^{W33}, —Si(R^{W34})₃, —N(R^{W35})₂, or a group having a phosphorus atom, R^{W32} and R^{W33} each independently represent a substituent, R^{W34} and R^{W35} each independently represent a hydrogen atom or a substituent, Z³¹ represents an oxygen atom, a sulfur atom, a selenium atom, =NR^{Z31}, or =C(R^{Z32})(R^{Z33}), and R^{Z31} to R^{Z33} each independently represent a hydrogen atom or a substituent.

2. The photoelectric conversion element according to claim 1,
wherein D¹ is a group represented by Formula (D-1), 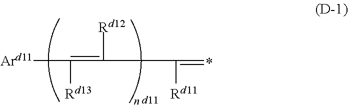
in Formula (D-1), * represents a bonding position, Ar^{d11} represents a substituent having an aromatic ring, R^{d11} to R^{d13} each independently represent a hydrogen atom or a substituent, and n^{d11} represents an integer of 0 to 5.

3. The photoelectric conversion element according to claim 2,
wherein Ar^{d11} is a group represented by any of Formulae (Ar-1) to (Ar-9), 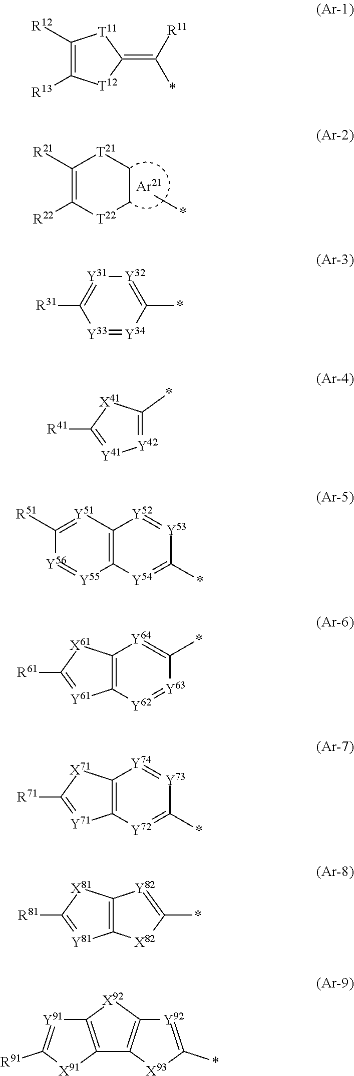
in Formula (Ar-1), * represents a bonding position, R¹¹ to R¹³ each independently represent a hydrogen atom or a substituent, at least two of R¹¹, R¹², or R¹³ may be bonded to each other to form a ring, T¹¹ and T¹² each independently represent an oxygen atom, a sulfur atom, a selenium atom, —NR¹⁴—, or —C(R¹⁵)(R¹⁶)—, and R¹⁴ to R¹⁶ each independently represent a hydrogen atom, an alkyl group which may have a substituent, or an aromatic ring group which may have a substituent,
in Formula (Ar-2), * represents a bonding position, Ar²¹ represents an aromatic ring which includes 2 or more carbon atoms and may have a substituent, R²¹ and R²² each independently represent a hydrogen atom or a substituent, R²¹ and R²² may be bonded to each other to form a ring, T²¹ and T²² each independently represent an oxygen atom, a sulfur atom, a selenium atom, —C(R²³)(R²⁴)—, —Si(R²⁵)(R²⁶)—, —NR²⁷—, or >C═R²⁸, R²³ to R²⁷ each independently represent a hydrogen atom or a substituent, R²³ and R²⁴, or R²⁵ and R²⁶ may be bonded to each other to form a ring, R²⁸ represents an oxygen atom, a sulfur atom, or =C(R²⁹)(R³⁰), R²⁹ and R³⁰ each independently represent a hydrogen atom or a substituent, and at least one of R²⁹ or R³⁰ and at least one of Ar²¹, R²¹, or R²² may be bonded to each other to form a ring,
in Formula (Ar-3), * represents a bonding position, R³¹ represents a hydrogen atom or a substituent, Y³¹ to Y³⁴ each independently represent —CR³²═ or a nitrogen atom, R³² represents a hydrogen atom, a halogen atom, a trifluoromethyl group, or a cyano group, and in a case where at least two of Y³¹, . . . , or Y³⁴ are —CR³²═, R³²'s may be bonded to each other to form a ring,
in Formula (Ar-4), * represents a bonding position, X⁴¹ represents an oxygen atom, a sulfur atom, a selenium atom, or —NR⁴²—, R⁴¹ and R⁴² each independently represent a hydrogen atom or a substituent, Y⁴¹ and Y⁴² each independently represent —CR⁴³═ or a nitrogen atom, R⁴³ represents a hydrogen atom, a halogen atom, a trifluoromethyl group, or a cyano group, and in a case where Y⁴¹ and Y⁴² are —CR⁴³═, R⁴³'s may be bonded to each other to form a ring,
in Formula (Ar-5), * represents a bonding position, R⁵¹ represents a hydrogen atom or a substituent, Y⁵¹ to Y⁵⁶ each independently represent —CR⁵²═ or a nitrogen atom, R⁵² represents a hydrogen atom, a halogen atom, a trifluoromethyl group, or a cyano group, and in a case where at least two of Y⁵¹, . . . , or Y⁵⁶ are —CR⁵²═, R⁵²'s may be bonded to each other to form a ring,
in Formula (Ar-6), * represents a bonding position, X⁶¹ represents an oxygen atom, a sulfur atom, a selenium atom, or —NR⁶²—, R⁶¹ and R⁶² each independently represent a hydrogen atom or a substituent, Y⁶¹ to Y⁶⁴ each independently represent —CR⁶³═ or a nitrogen atom, R⁶³ represents a hydrogen atom, a halogen atom, a trifluoromethyl group, or a cyano group, and in a case where at least two of Y⁶¹, . . . , or Y⁶⁴ are —CR⁶³═, R⁶³'s may be bonded to each other to form a ring,
in Formula (Ar-7), * represents a bonding position, X⁷¹ represents an oxygen atom, a sulfur atom, a selenium atom, or —NR⁷²—, R⁷¹ and R⁷² each independently represent a hydrogen atom or a substituent, Y⁷¹ to Y⁷⁴ each independently represent —CR⁷³═ or a nitrogen atom, R⁷³ represents a hydrogen atom, a halogen atom, a trifluoromethyl group, or a cyano group, and in a case where at least two of Y⁷¹, . . . , or Y⁷⁴ are —CR⁷³═, R⁷³'s may be bonded to each other to form a ring,
in Formula (Ar-8), * represents a bonding position, X⁸¹ and X⁸² each independently represent an oxygen atom, a sulfur atom, a selenium atom, or —NR⁸²—, R⁸¹ and R⁸² each independently represent a hydrogen atom or a substituent, Y⁸¹ and Y⁸² each independently represent —CR⁸³═ or a nitrogen atom, and R⁸³ represents a hydrogen atom, a halogen atom, a trifluoromethyl group, or a cyano group,
in Formula (Ar-9), * represents a bonding position, X⁹¹ to X⁹³ each independently represent an oxygen atom, a sulfur atom, a selenium atom, or —NR⁹²—, R⁹¹ and R⁹² each independently represent a hydrogen atom or a substituent, Y⁹¹ and Y⁹² each independently represent —CR⁹³═ or a nitrogen atom, and R⁹³ represents a hydrogen atom, a halogen atom, a trifluoromethyl group, or a cyano group.

4. The photoelectric conversion element according to claim 2,
wherein Ar^{d11} is a group represented by Formula (Ar-10), 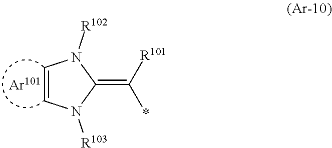
in Formula (Ar-10), * represents a bonding position, R¹⁰¹ represents a hydrogen atom or a substituent, R¹⁰² and R¹⁰³ each independently represent a hydrogen atom, an alkyl group which may have a substituent, or an aromatic ring group which may have a substituent, and Ar¹⁰¹ represents an aromatic ring which includes 2 or more carbon atoms and may have a substituent.

5. The photoelectric conversion element according to claim 1,
wherein Z¹¹, Z¹², Z²¹, Z²², and Z³¹ are each independently an oxygen atom or a sulfur atom.

6. A photoelectric conversion element comprising, in the following order:
a conductive film;
a photoelectric conversion film; and
a transparent conductive film,
wherein the photoelectric conversion film contains a compound represented by Formula (2),
D²=A² (2)
in Formula (2), A² represents a group represented by any of Formulae (A-1) to (A-4) and D² represents a group represented by Formula (D-2), 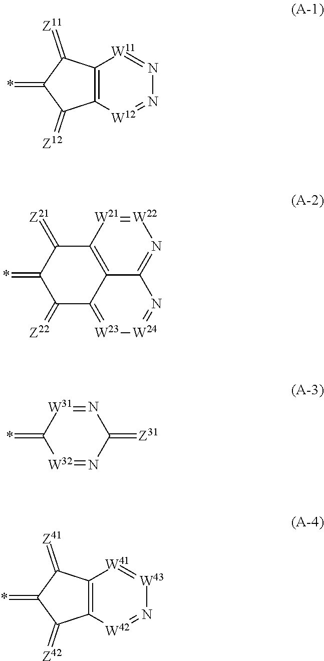
in Formula (A-1), * represents a bonding position, W¹¹ and W¹² each independently represent —CR^{W11}═ or a nitrogen atom, R^{W11} represents a hydrogen atom or a substituent, Z¹¹ and Z¹² each independently represent an oxygen atom, a sulfur atom, a selenium atom, =NR^{Z11}, or —C(R^{Z12})(R^{Z13}), and R^{Z11} to R^{Z13} each independently represent a hydrogen atom or a substituent,
in Formula (A-2), * represents a bonding position, W²¹ to W²⁴ each independently represent —CR^{W21}═ or a nitrogen atom, R^{W21} represents a hydrogen atom or a substituent, Z²¹ and Z²² each independently represent an oxygen atom, a sulfur atom, a selenium atom, =NR^{Z21}, or =C(R^{Z22})(R^{Z23}), and R^{Z21} to R^{Z23} each independently represent a hydrogen atom or a substituent,
in Formula (A-3), * represents a bonding position, W³¹ and W³² each independently represent —CR^{W31}═ or a nitrogen atom, R^{W31} represents a hydrogen atom, a halogen atom, a cyano group, an aromatic ring group which may have a substituent, an aliphatic hydrocarbon group which may have a substituent, —OR^{W32}, —SR^{W33}, —Si(R^{W34})₃, —N(R^{W35})₂, or a group having a phosphorus atom, R^{W32} and R^{W33} each independently represent a substituent, R^{W34} and R^{W35} each independently represent a hydrogen atom or a substituent, Z³¹ represents an oxygen atom, a sulfur atom, a selenium atom, =NR^{Z31}, or =C(R^{Z32})(R^{Z33}), and R^{Z31} to R^{Z33} each independently represent a hydrogen atom or a substituent,
in Formula (A-4), * represents a bonding position, W⁴¹ to W⁴³ each independently represent —CR^{W41}═ or a nitrogen atom, R^{W41} represents a hydrogen atom or a substituent, Z⁴¹ and Z⁴² each independently represent an oxygen atom, a sulfur atom, a selenium atom, =NR^{Z41}, or =C(R^{Z42})(R^{Z43}), and R^{Z41} to R^{Z43} each independently represent a hydrogen atom or a substituent, 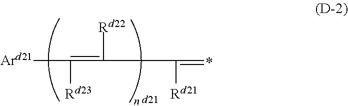
in Formula (D-2), * represents a bonding position, Ar^{d21} represents a group represented by Formula (Ar-1), R^{d21} to R^{d23} each independently represent a hydrogen atom or a substituent, and n^{d21} represents an integer of 0 to 5, 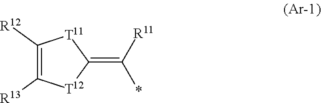
in Formula (Ar-1), * represents a bonding position, R¹¹ to R¹³ each independently represent a hydrogen atom or a substituent, at least two of R¹¹, R¹², or R¹³ may be bonded to each other to form a ring, T¹¹ and T¹² each independently represent an oxygen atom, a sulfur atom, a selenium atom, —NR¹⁴—, or —C(R¹⁵)(R¹⁶)—, and R¹⁴ to R¹⁶ each independently represent a hydrogen atom, an alkyl group which may have a substituent, or an aromatic ring group which may have a substituent.

7. The photoelectric conversion element according to claim 6,
wherein Ar^{d21} is a group represented by Formula (Ar-10), 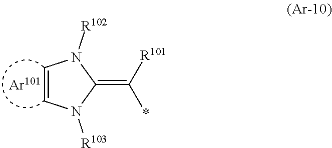
in Formula (Ar-10), * represents a bonding position, R¹⁰¹ represents a hydrogen atom or a substituent, R¹⁰² and R¹⁰³ each independently represent a hydrogen atom, an alkyl group which may have a substituent, or an aromatic ring group which may have a substituent, and Ar¹⁰¹ represents an aromatic ring which includes 2 or more carbon atoms and may have a substituent.

8. The photoelectric conversion element according to claim 6,
wherein Z¹¹, Z¹², Z²¹, Z²², Z³¹, Z⁴¹, and Z⁴² are each independently an oxygen atom or a sulfur atom.

9. The photoelectric conversion element according to claim 1, further comprising:
one or more interlayers between the conductive film and the transparent conductive film, in addition to the photoelectric conversion film.

10. An imaging element comprising:
the photoelectric conversion element according to claim 1.

11. An optical sensor comprising:
the photoelectric conversion element according to claim 1.

12. A compound represented by Formula (1),
$${D}^{1} = {A}^{1} \qquad ( 1 )$$
in Formula (1), A¹ represents a group represented by any of Formulae (A-1) to (A-3), and D¹ represents a divalent organic group, 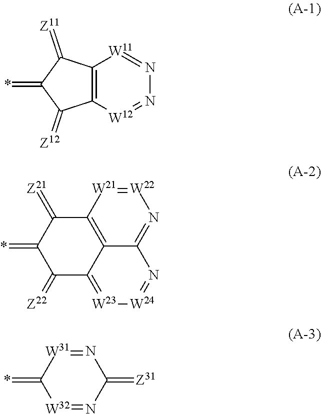
in Formula (A-1), * represents a bonding position, W¹¹ and W¹² each independently represent —CR^{W11}═ or a nitrogen atom, R^{W11} represents a hydrogen atom or a substituent, Z¹¹ and Z¹² each independently represent an oxygen atom, a sulfur atom, a selenium atom, =NR^{Z11}, or =C(R^{Z12})(R^{Z13}), and R^{Z11} to R^{Z13} each independently represent a hydrogen atom or a substituent,
in Formula (A-2), * represents a bonding position, W²¹ to W²⁴ each independently represent —CR^{W21}═ or a nitrogen atom, R^{W21} represents a hydrogen atom or a substituent, Z²¹ and Z²² each independently represent an oxygen atom, a sulfur atom, a selenium atom, =NR^{Z21}, or =C(R^{Z22})(R^{Z23}), and R^{Z21} to R^{Z23} each independently represent a hydrogen atom or a substituent,
in Formula (A-3), * represents a bonding position, W³¹ and W³² each independently represent —CR^{W31}═ or a nitrogen atom, where W³¹ and W³² are each —CR^{W31}═ or one of W³¹ and W³² is —CR^{W31}═ and the other thereof is a nitrogen atom, R^{W31} represents a hydrogen atom, a halogen atom, a cyano group, an aromatic ring group which may have a substituent, an aliphatic hydrocarbon group which may have a substituent, —OR^{W32}, —SR^{W33}, —Si(R^{W34})₃, —N(R^{W35})₂, or a group having a phosphorus atom, R^{W32} and R^{W33} each independently represent a substituent, R^{W34} and R^{W35} each independently represent a hydrogen atom or a substituent, Z³¹ represents an oxygen atom, a sulfur atom, a selenium atom, =NR^{Z31}, or =C(R^{Z32})(R^{Z33}), and R^{Z31} to R^{Z33} each independently represent a hydrogen atom or a substituent.

13. The compound according to claim 12,
wherein D¹ is a group represented by Formula (D-1), 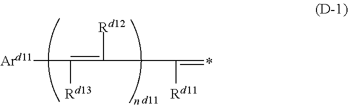
in Formula (D-1), * represents a bonding position, Ar^{d11} represents a substituent having an aromatic ring, R^{d11} to R^{d13} each independently represent a hydrogen atom or a substituent, and n^{d11} represents an integer of 0 to 5.

14. The compound according to claim 13,
wherein Ar^{d11} is a group represented by any of Formulae (Ar-1) to (Ar-9), 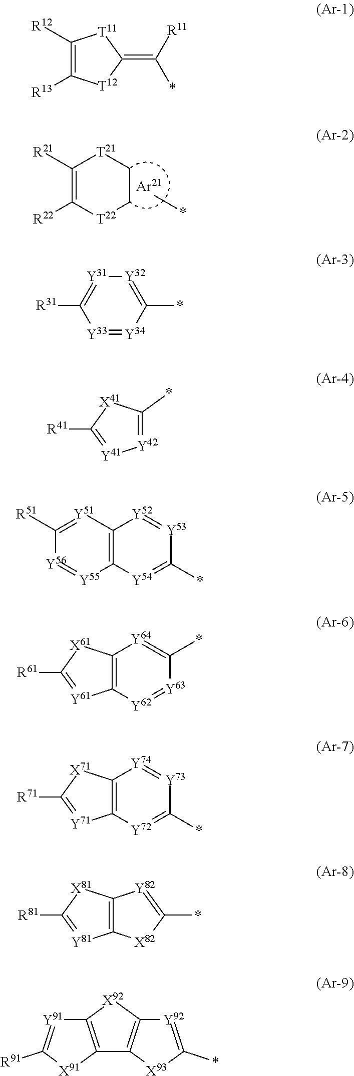
in Formula (Ar-1), * represents a bonding position, R¹¹ to R¹³ each independently represent a hydrogen atom or a substituent, at least two of R¹¹, R¹², or R¹³ may be bonded to each other to form a ring, T¹¹ and T¹² each independently represent an oxygen atom, a sulfur atom, a selenium atom, —NR¹⁴—, or —C(R¹⁵)(R¹⁶)—, and R¹⁴ to R¹⁶ each independently represent a hydrogen atom, an alkyl group which may have a substituent, or an aromatic ring group which may have a substituent,
in Formula (Ar-2), * represents a bonding position, Ar²¹ represents an aromatic ring which includes 2 or more carbon atoms and may have a substituent, R²¹ and R²² each independently represent a hydrogen atom or a substituent, R²¹ and R²² may be bonded to each other to form a ring, T²¹ and T²² each independently represent an oxygen atom, a sulfur atom, a selenium atom, —C(R²³)(R²⁴)—, —Si(R²⁵)(R²⁶)—, —NR²⁷—, or >C=R²⁸, R²³ to R²⁷ each independently represent a hydrogen atom or a substituent, R²³ and R²⁴, or R²⁵ and R²⁶ may be bonded to each other to form a ring, R²⁸ represents an oxygen atom, a sulfur atom, or =C(R²⁹)(R³⁰), R²⁹ and R³⁰ each independently represent a hydrogen atom or a substituent, and at least one of R²⁹ or R³⁰ and at least one of Ar²¹, R²¹, or R²² may be bonded to each other to form a ring,
in Formula (Ar-3), * represents a bonding position, R³¹ represents a hydrogen atom or a substituent, Y³¹ to Y³⁴ each independently represent —CR³²═ or a nitrogen atom, R³² represents a hydrogen atom, a halogen atom, a trifluoromethyl group, or a cyano group, and in a case where at least two of Y³¹, . . . , or Y³⁴ are —CR³²═, R³²'s may be bonded to each other to form a ring,
in Formula (Ar-4), * represents a bonding position, X⁴¹ represents an oxygen atom, a sulfur atom, a selenium atom, or —NR⁴²—, R⁴¹ and R⁴² each independently represent a hydrogen atom or a substituent, Y⁴¹ and Y⁴² each independently represent —CR⁴³═ or a nitrogen atom, R⁴³ represents a hydrogen atom, a halogen atom, a trifluoromethyl group, or a cyano group, and in a case where Y⁴¹ and Y⁴² are —CR⁴³═, R⁴³'s may be bonded to each other to form a ring,
in Formula (Ar-5), * represents a bonding position, R⁵¹ represents a hydrogen atom or a substituent, Y⁵¹ to Y⁵⁶ each independently represent —CR⁵²═ or a nitrogen atom, R⁵² represents a hydrogen atom, a halogen atom, a trifluoromethyl group, or a cyano group, and in a case where at least two of Y⁵¹, . . . , or Y⁵⁶ are —CR⁵²—, R⁵²'s may be bonded to each other to form a ring,
in Formula (Ar-6), * represents a bonding position, X⁶¹ represents an oxygen atom, a sulfur atom, or a selenium atom or —NR⁶²—, R⁶¹ and R⁶² each independently represent a hydrogen atom or a substituent, Y⁶¹ to Y⁶⁴ each independently represent —CR⁶³═ or a nitrogen atom, R⁶³ represents a hydrogen atom, a halogen atom, a trifluoromethyl group, or a cyano group, and in a case where at least two of Y⁶¹, . . . , or Y⁶⁴ are —CR⁶³═, R⁶³'s may be bonded to each other to form a ring,
in Formula (Ar-7), * represents a bonding position, X⁷¹ represents an oxygen atom, a sulfur atom, or a selenium atom or —NR⁷²—, R⁷¹ and R⁷² each independently represent a hydrogen atom or a substituent, Y⁷¹ to Y⁷⁴ each independently represent —CR⁷³═ or a nitrogen atom, R⁷³ represents a hydrogen atom, a halogen atom, a trifluoromethyl group, or a cyano group, and in a case where at least two of Y⁷¹, . . . , or Y⁷⁴ are —CR⁷³═, R⁷³'s may be bonded to each other to form a ring,
in Formula (Ar-8), * represents a bonding position, X⁸¹ and X⁸² each independently represent an oxygen atom, a sulfur atom, a selenium atom, or —NR⁸²—, R⁸¹ and R⁸² each independently represent a hydrogen atom or a substituent, Y⁸¹ and Y⁸² each independently represent —CR⁸³═ or a nitrogen atom, and R⁸³ represents a hydrogen atom, a halogen atom, a trifluoromethyl group, or a cyano group,
in Formula (Ar-9), * represents a bonding position, X⁹¹ to X⁹³ each independently represent an oxygen atom, a sulfur atom, a selenium atom, or —NR⁹²—, R⁹¹ and R⁹² each independently represent a hydrogen atom or a substituent, Y⁹¹ and Y⁹² each independently represent —CR⁹³═ or a nitrogen atom, and R⁹³ represents a hydrogen atom, a halogen atom, a trifluoromethyl group, or a cyano group.

15. The compound according to claim 13,
wherein Ar^{d11} is a group represented by Formula (Ar-10), 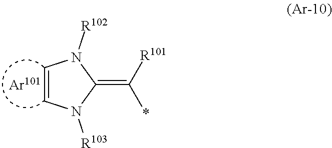
in Formula (Ar-10), * represents a bonding position, R¹⁰¹ represents a hydrogen atom or a substituent, R¹⁰² and R¹⁰³ each independently represent a hydrogen atom, an alkyl group which may have a substituent, or an aromatic ring group which may have a substituent, and Ar¹⁰¹ represents an aromatic ring which includes 2 or more carbon atoms and may have a substituent.

16. The compound according to claim 12,
wherein Z¹¹, Z¹², Z²¹, Z²², and Z³¹ are each independently an oxygen atom or a sulfur atom.

17. A compound represented by Formula (2),
$${D}^{2} = {A}^{2} \qquad ( 2 )$$
in Formula (2), A² represents a group represented by any of Formulae (A-1) to (A-4), and D² represents a group represented by Formula (D-2), 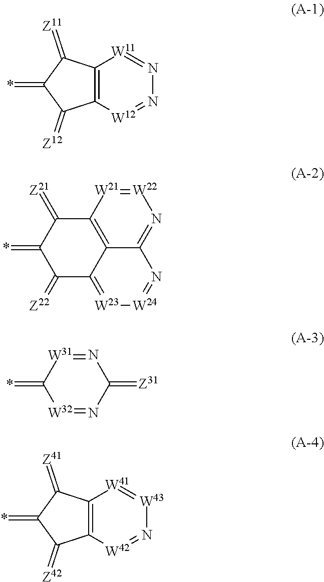
in Formula (A-1), * represents a bonding position, W¹¹ and W¹² each independently represent —CR^{W11}═ or a nitrogen atom, R^{W11} represents a hydrogen atom or a substituent, Z¹¹ and Z¹² each independently represent an oxygen atom, a sulfur atom, a selenium atom, =NR^{Z11}, or =C(R^{Z12})(R^{Z13}), and R^{Z11} to R^{Z13} each independently represent a hydrogen atom or a substituent,
in Formula (A-2), * represents a bonding position, W²¹ to W²⁴ each independently represent —CR^{W21}═ or a nitrogen atom, R^{W21} represents a hydrogen atom or a substituent, Z²¹ and Z²² each independently represent an oxygen atom, a sulfur atom, a selenium atom, =NR^{Z21}, or ═C(R^{Z22})(R^{Z23}), and R^{Z21} to R^{Z23} each independently represent a hydrogen atom or a substituent,
in Formula (A-3), * represents a bonding position, W³¹ and W³² each independently represent —CR^{W31}═ or a nitrogen atom, R^{W31} represents a hydrogen atom, a halogen atom, a cyano group, an aromatic ring group which may have a substituent, an aliphatic hydrocarbon group which may have a substituent, —OR^{W32}, —SR^{W33}, —Si(R^{W34})₃, —N(R^{W35})₂, or a group having a phosphorus atom, R^{W32} and R^{W33} each independently represent a substituent, R^{W34} and R^{W35} each independently represent a hydrogen atom or a substituent, Z³¹ represents an oxygen atom, a sulfur atom, a selenium atom, =NR^{Z31}, or —C(R^{Z32})(R^{Z33}), and R^{Z31} to R^{Z33} each independently represent a hydrogen atom or a substituent,
in Formula (A-4), * represents a bonding position, W⁴¹ to W⁴³ each independently represent —CR^{W41}═ or a nitrogen atom, R^{W41} represents a hydrogen atom or a substituent, Z⁴¹ and Z⁴² each independently represent an oxygen atom, a sulfur atom, a selenium atom, =NR^{Z41}, or =C(R^{Z42})(R^{Z43}), and R^{Z41} to R^{Z43} each independently represent a hydrogen atom or a substituent, 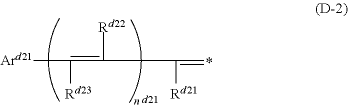
in Formula (D-2), * represents a bonding position, Ar^{d21} represents a group represented by Formula (Ar-1), R^{d21} to R^{d23} each independently represent a hydrogen atom or a substituent, and n^{d21} represents an integer of 0 to 5, 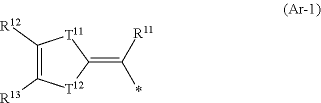
in Formula (Ar-1), * represents a bonding position, R¹¹ to R¹³ each independently represent a hydrogen atom or a substituent, at least two of R¹¹, R¹², or R¹³ may be bonded to each other to form a ring, T¹¹ and T¹² each independently represent an oxygen atom, a sulfur atom, a selenium atom, —NR¹⁴—, or —C(R¹⁵)(R¹⁶)—, and R¹⁴ to R¹⁶ each independently represent a hydrogen atom, an alkyl group which may have a substituent, or an aromatic ring group which may have a substituent.

18. The compound according to claim 17,
wherein Ar^{d21} is a group represented by Formula (Ar-10), 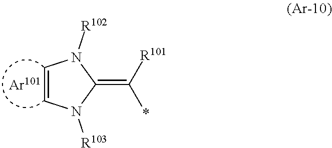
in Formula (Ar-10), * represents a bonding position, R¹⁰¹ represents a hydrogen atom or a substituent, R¹⁰² and R¹⁰³ each independently represent a hydrogen atom, an alkyl group which may have a substituent, or an aromatic ring group which may have a substituent, and Ar¹⁰¹ represents an aromatic ring which includes 2 or more carbon atoms and may have a substituent.

19. The compound according to claim 17,
wherein Z¹¹, Z¹², Z²¹, Z²², Z³¹, Z⁴¹, and Z⁴² are each independently an oxygen atom or a sulfur atom.
